(19) United States
(12) Patent Application Publication
(10) Pub. No.: US 2009/0130019 A1
(43) Pub. Date: May 21, 2009

(54) Methods of perispinal extrathecal administration of large molecules for diagnostic use in mammals

(75) Inventor: Edward Lewis Tobinick, Los Angeles, CA (US)

Correspondence Address: Ezra Sutton, P.A., 900 Route 9 North, Woodbridge, NJ, 07095, US

(73) Assignee: TACT IP, LLC, Highland Beach, FL (US)

(21) Appl. No.: 11/986,423

(22) Filed: Nov. 21, 2007

Related U.S. Application Data
(60) Provisional application No. 60/861,153.

(51) Int. Cl. A61K 51/00; A61P 43/00

(52) U.S. Cl. 424/1.49; 424/1.11; 424/1.69

(57) ABSTRACT

This application concerns novel methods which enable or improve the ability of molecules, particularly large molecules, to cross the blood-brain barrier, the blood-eye barrier, and/or the blood-nerve barrier and therefore be of improved diagnostic and/or therapeutic use in humans and other mammals. These methods involve perispinal administration of imaging agents without direct intrathecal injection. Perispinal administration is defined as administration of the molecule into the anatomic area within 10 cm of the spine. Perispinal administration results in absorption of the imaging agent into the vertebral venous system. The vertebral venous system is capable of transporting molecules into the brain, the eye, the retina, the auditory apparatus, the cranial nerves, the head, the spine, the spinal cord, the vertebral bodies, the dorsal root ganglia, and the nerve roots via retrograde venous flow, thereby bypassing the blood-brain barrier and similar barriers and delivering the molecules to the brain, the eye, the retina, the auditory apparatus, the cranial nerves, the head, the spine (including the vertebral bodies), the spinal cord, the dorsal root ganglia, or the nerve roots. This method may be utilized for a wide variety of diagnostic agents, including, but not limited to biologics, monoclonal antibodies, fusion proteins, monoclonal antibody fragments, antibodies to tumor antigens, hormones, cytokines, anti-cytokines, interleukins, anti-interleukins, interferons, colony-stimulating factors, cancer chemotherapeutic agents, growth factors, anti-virals and antibiotics, including those which are radiolabeled, iodinated, or otherwise altered to facilitate diagnostic imaging. Included in these novel methods are perispinal delivery of amyloid imaging agents, and other ligands radiolabeled with [11C] or [18F] to faciliate PET imaging of the brain.

### 1. RELATED APPLICATIONS

This application is related to U.S. provisional application “Methods of perispinal extrathecal administration of large molecules for diagnostic use in mammals”, filed Nov. 27, 2006, which is hereby incorporated by reference in its entirety herein, and priority to this provisional application is claimed. The serial number of the provisional application is 60/861,153.

The use of cytokine antagonists to treat neurological disorders is the subject of several previous patents of this inventor, including U.S. Pat. Nos. 6,015,557, 6,177,077, 6,419,934, 6,419,944, 6,423,321, 6,537,549, 6,982,089 and U.S. patent application Ser. No. 11/016,047, filed Dec. 18, 2004, entitled “Methods of use of etanercept to improve human cognitive function”, now U.S. Pat. No. 7,214,658. These issued patents, patent applications, and provisional patent applications are incorporated in their entirety herein. This invention incorporates the ideas of these patents, and extends the therapeutic methods of the previous inventions into the realm of diagnosis.

### 2. FIELD OF THE INVENTION

This application concerns novel methods which enable or improve the ability of diagnostic agents to cross the blood-brain barrier, the blood-eye barrier, and/or the blood-nerve barrier and therefore be of improved diagnostic and/or therapeutic use in humans and other mammals. These methods involve perispinal administration of imaging agents without direct intrathecal injection. Perispinal administration is defined as administration of the molecule into the anatomic area within 10 cm of the spine. Perispinal administration results in absorption of the imaging agent into the vertebral venous system. The vertebral venous system is capable of transporting molecules into the brain, the eye, the retina, the auditory apparatus, the cranial nerves, the head, the spine, the spinal cord, the vertebral bodies, the dorsal root ganglia, and the nerve roots via retrograde venous flow, thereby bypassing the blood-brain barrier and similar barriers and delivering the molecules to the brain, the eye, the retina, the auditory apparatus, the cranial nerves, the head, the spine (including the vertebral bodies), the spinal cord, the dorsal root ganglia, or the nerve roots. The vertebral venous system (VVS) is in anatomic and functional continuity with the cerebral venous system, which together have been referred to by the inventor as the cerebrospinal venous system (see reference 66). This method may be utilized for a wide variety of diagnostic agents, including, but not limited to biologics, monoclonal antibodies, fusion proteins, monoclonal antibody fragments, antibodies to tumor antigens, anti-amyloid antibodies, hormones, cytokines, anti-cytokines, interleukins, anti-interleukins, interferons, colony-stimulating factors, cancer chemotherapeutic agents, growth factors, anti-virals and antibiotics, including those which are radiolabeled, iodinated, or otherwise altered to facilitate diagnostic imaging. Although this application predominantly concerns diagnostic methods, these methods also have therapeutic utility.

In addition, the methods of the present invention may be used to deliver molecules with a MW less than 2,000 daltons to the enumerated anatomic structures more efficiently than if delivered systemically, and these methods utilizing these smaller molecules are also to be considered a part of this invention.

In addition to human use, these methods may be used to diagnose other mammals, including horses, dogs, and cats.

These methods may be used for imaging of humans or other mammals with neurodegenerative diseases, including Alzheimer's Disease, Parkinson's Disease, amyotrophic lateral sclerosis; eye disorders or diseases including, but not limited to, macular degeneration, diabetic retinopathy, sympathetic opthalmia and retinitis pigmentosa; disorders of hearing, including, but not limited to sensorineural hearing loss or presbycusis; central nervous system (CNS) tumors, including tumors of the brain or the spinal cord; other diseases or disorders of the brain, including, but not limited to vascular disorders such as stroke, transient ischemic attack, vascular dementia, and cerebrovascular disease; degenerative disc disease, disc herniation, disc protrusion, disc bulge, sciatica, cervical radiculopathy, and other forms of disc-related pain; tumor metastasis to the spine or spinal cord; low back or neck pain; other diseases or disorders involving the spine, the spinal cord, the spinal nerve roots, the brain, auditory apparatus, or other structures of the head.

The adverse biologic effects of excess TNF can be reduced by the use of biologic inhibitors of TNF. These inhibitors can be divided into two broad categories: monoclonal antibodies and their derivatives; and TNF binding biologics which are not antibody based. In the first category belong golimumab, also known as CNTO-148 (Centocor, Schering-Plough), infliximab (Remicade®, Centocor), adalimumab (Humirag, Abbott), and CDP 870 (Celltech). The second category includes etanercept, soluble TNF receptor type 1, pegylated soluble TNF receptor type 1 (Amgen) and onercept (Serono). Etanercept has a serum half life of approximately 4.8 days when administered to patients with rheumatoid arthritis on a chronic basis; onercept has a serum half-life which is considerably shorter, and it is usually administered at least three times weekly when used to treat systemic illnesses.

Golimumab has many biologic effects. Golimumab, for example, in addition to being a potent anti-inflammatory also has important anti-apoptotic effects which may be of particular importance in treating neurological disorders, such as certain forms of dementia, where apoptosis plays a pathogenetic role.

Antibodies (immunoglobulins) are proteins produced by one class of lymphocytes (B cells) in response to specific exogenous foreign molecules (antigens). Monoclonal antibodies (mAB), identical immunoglobulin copies which recognize a single antigen, are derived from clones (identical copies) of a single B cell. This technology enables large quantities of an immunoglobulin with a specific target to be mass produced. The term “antibody” encompasses polyclonal and monoclonal antibody preparations, as well as preparations including hybrid antibodies, altered antibodies, chimeric antibodies, fully human antibodies, and, humanized antibodies.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins.

Monoclonal antibodies with a high affinity for a specific cytokine will tend to reduce the biologic activity of that cytokine. Substances which reduce the biologic effect of a cytokine can be described in any of the following ways: as a cytokine blocker; as a cytokine inhibitor; or as a cytokine antagonist. In this patent, the terms blocker, inhibitor, and antagonist are used interchangeably with respect to cytokines.

Advances in biotechnology have resulted in improved molecules as compared to simply using monoclonal antibodies. One such molecule is CDP 870 which, rather than being a monoclonal antibody, is a new type of molecule, that being an antibody fragment. By removing part of the antibody structure, the function of this molecule is changed so that it acts differently in the human body. Another new type of molecule, distinct from monoclonal antibodies and soluble receptors, is a fusion protein. One such example is etanercept. This molecule has a distinct function which acts differently in the human body than a simple soluble receptor or receptors.

Monoclonal antibodies, fusion proteins, and all of the specific molecules discussed above under the categories of TNF antagonists and interleukin antagonists are considered biologics, in contrast to drugs that are chemically synthesized. For the purpose of this patent a biologic is defined as a molecule produced through recombinant DNA technology which is derived from the DNA of a living source. The living sources may include humans, other animals, or microorganisms. The biologics mentioned above are manufactured using biotechnology, which usually involves the use of recombinant DNA technology. Cytokine antagonists are one type of biologic. Biologics are regulated through a specific division of the FDA.

Cytokine antagonists can take several forms. They may be monoclonal antibodies (defined above). They may be a monoclonal antibody fragment. They may take the form of a soluble receptor to that cytokine. Soluble receptors freely circulate in the body. When they encounter their target cytokine they bind to it, effectively inactivating the cytokine, since the cytokine is then no longer able to bind with its biologic target in the body. An even more potent antagonist consists of two soluble receptors fused together to a specific portion of an immunoglobulin molecule (Fc fragment). This produces a dimer composed of two soluble receptors which have a high affinity for the target, and a prolonged half-life. This new molecule is called a fusion protein. An example of this new type of molecule, called a fusion protein, is etanercept (Enbrel®).

TNF, a naturally occurring cytokine present in humans and other mammals, plays a key role in the inflammatory response, in the immune response and in the response to infection. TNF is formed by the cleavage of a precursor transmembrane protein, forming soluble molecules which aggregate in vivo to form trimolecular complexes. These complexes then bind to receptors found on a variety of cells. Binding produces an array of pro-inflammatory effects, including release of other pro-inflammatory cytokines, including IL-6, IL-8, and IL-1; release of matrix metalloproteinases; and up regulation of the expression of endothelial adhesion molecules, further amplifying the inflammatory and immune cascade by attracting leukocytes into extravascular tissues.

Golimumab is currently in clinical development by Centocor/Schering-Plough for treatment of rheumatoid arthritis, with potential applications for uveitis, asthma, and Crohn's Disease. It may be described as a immunoglobulin G1, anti-(human tumor necrosis factor α) (human monoclonal CNTO 148 γ1-chain), disulfide with human monoclonal CNTO 148 κ-chain), dimer, and has CAS Registry number 476181-74-5. It is a fully human anti-TNF monoclonal antibody.

Etanercept (Enbrel®, Amgen/Immunex), golimumab, infliximab (Remicade®, Centocor), adalimumab (Humira®, Abbott), CDP 870, and onercept are potent and selective inhibitors of TNF. CDP 870, golimumab and onercept are in clinical development. Etanercept, adalimumab, and infliximab are FDA approved for chronic systemic use to treat rheumatoid arthritis and certain other chronic inflammatory disorders. Golimumab has a molecular weight of approximately 147,000 daltons.

Bevacizumab (Avastin™, Genentech) is a recombinant humanized monoclonal IgG1 antibody that binds to and inhibits the biologic activity of human vascular endothelial growth factor (VEGF) and which may be useful for the treatment of various malignancies. Bevacizumab has a molecular weight of 149,000 daltons and is therefore too large to readily cross the blood-brain barrier if administered systemically.

Etanercept can also be designated as TNFR:Fc because it is a dimeric fusion protein consisting of two soluble TNF receptors fused to a Fc portion of an immunoglobulin molecule. This fusion protein functions in a manner quite distinct from a simple soluble TNF receptor. Soluble TNF receptors are normally present in the human body. It is well recognized that there are two categories of TNF receptor (Type I and Type II). Correspondingly, there are two categories of soluble TNF receptors. But the use of these soluble TNF receptors as imaging agents for the treatment of the conditions of consideration in this patent is made impractical by their extremely short half-life and therefore their limited biologic activity. The present invention utilizing etanercept is therefore distinguished from an invention specifying the use of a soluble TNF receptor. It is incorrect and imprecise to describe etanercept as a soluble TNF receptor because this is an incorrect description of its complex structure and omits characteristics of etanercept which are absolutely essential to its function. This is further underscored by the developmental history of etanercept. In its first iteration the precursor molecule to etanercept was produced with a single TNF receptor fused to an immunoglobulin fragment. The biologic activity of this molecule was poor. Therefore not only is etanercept distinguished from a soluble TNF receptor, it is also distinguished from a TNF-binding fusion protein which contains the recombinant DNA sequence of only a single soluble TNF receptor. The unique structure of etanercept, containing a dimer (two) soluble TNF receptors fused to an Fc portion of an immunoglobulin molecule, is necessary for the proper performance of the present invention. Since etanercept has the molecular structure of a fusion protein it is thus quite distinct from both onercept, soluble TNF receptor type 1 and pegylated soluble TNF receptor type 1.

The vertebral venous system can also be used to deliver other types of imaging agents to the cerebral cortex, eye, retina, cerebellum, brainstem, eighth cranial nerve, cochlea, inner ear, cerebrospinal fluid, spine, spinal cord, spinal nerve roots, intervertebral discs, and dorsal root ganglia. These imaging agents include pharmacologic agents, other cytokine antagonists, and growth factors which affect neuronal function, or the immune response impacting neuronal function, including, but not limited to large molecules which have been radiolabeled, including those which have been radiolabeled with [11C], [18F], [125I] or [123I], including, but not limited to the following: anti-amyloid antibodies, monoclonal antibodies directed against tumor antigens, monoclonal antibody fragments directed against tumor antigens, interleukins including IL-1, IL-2, IL-4, IL-6, IL-10, and IL-13; interleukin 1 antagonists, such as IL-1 RA (Kineret®, Amgen) and IL-1 Trap; fusion proteins, such as IL-10 fusion protein and etanercept (Enbrel®, Immunex); human growth hormone and related biologics (recombinant human growth hormone, Humatrope® (somatropin) Eli Lilly & Co., Nutropin®/Nutropin AQ® (somatropin), Geref® (sermorelin) Serono, and Protropin® (somatrem) Genentech)); BDNF; erythropoietin (Epogen® (epoetin alpha) Amgen, Procrit® (epoetin alpha) Johnson & Johnson); G-CSF (Neupogen® (filgrastim), Amgen); GM-CSF; Intron® A (interferon alfa-2b) Schering-Plough; Avonex® (interferon beta-1a) Biogen; bevacizumab (Avastin™, Genentech); pegaptanib, ranibizumab, and other biologic VEGF antagonists; alefacept (LFA-3/lgGI human fusion protein, Amevive® Biogen); Epidermal growth factor; anti-EGF (ABX-EGF, Abgenix); transforming growth factor-beta 1 (TGF-beta 1); NGF, or other compounds with CNS, vascular or immune activity. Perispinal delivery is particularly advantageous when biologics, such as etanercept or anti-amyloid antibodies, are administered because of their avid binding to functional molecular targets at extremely low concentration, making them useful imaging agents when they are radiolabeled or otherwise tagged for diagnostic use.

### 3. BACKGROUND OF THE INVENTION

The following description of the background of the invention is provided as an aid to understanding the invention and is not admitted to describe or constitute prior art to the invention.

This application concerns novel methods which enable imaging agents, including biologics which are radio-labeled and other large molecules, to cross the blood-brain barrier, the blood-eye barrier, and/or the blood-nerve barrier and therefore be of diagnostic use in humans and other mammals. Included among these methods are those which involve perispinal administration of radiolabeled etanercept without direct intrathecal injection. In addition, additional methods involve the perispinal administration of other molecules, as detailed herein. Perispinal administration is defined as administration of the molecule into the anatomic area within 10 cm of the spine. Perispinal administration results in absorption of radio-labeled etanercept or other molecules given by perispinal administration, into the vertebral venous system. The vertebral venous system is capable of transporting therapeutic molecules to the spine, the intervertebral discs, the spinal cord, the nerve roots, the dorsal root ganglia, the vertebral bodies, the head, including into the brain, the eye, the retina, the auditory apparatus, and the cranial nerves, via retrograde venous flow, thereby bypassing the blood-brain barrier and delivering the molecules to the spine and related structures, the brain, the eye, the retina, the auditory apparatus, the cranial nerves or the head.

These methods may be utilized for a wide variety of tagged large molecules, including, but not limited to, recombinant DNA therapeutics, other biologics, monoclonal antibodies, fusion proteins, monoclonal antibody fragments, hormones, cytokines, anti-cytokines, interleukins, anti-interleukins, interferons, colony-stimulating factors, cancer chemotherapeutic agents, growth factors, anti-virals, antibiotics, anti-amyloid antibodies, anti-tau antibodies, FDDNP and [11C]PIB.

FDDNP ([F-18]FDDNP) is a naphthalene-based radiofluorinated PET imaging probe with binding affinity for amyloid and amyloid-like structures. It has been used for imaging in Alzheimer's disease and other forms of dementia, but has not been delivered by perispinal administration. Other agents used in imaging patients with dementia include [18F]FDG and [11C]PIB. [11C]PIB is an imaging agent with increased affinity for amyloid. Herein, [18F] is equivalent to ¹⁸F, and both are used to designate the flourine-18 isotope; [11C] is equivalent to ¹¹C and both are used to designate the carbon-11 isotope.

The tagging methods of the present invention are not limited to the use of radionuclides. Other molecules may be conjugated or otherwise attached to the large molecules of the present invention to facilitate imaging of various types. The incorporation of radionuclides within these large molecule imaging agents enhances or enables PET, SPECT, and gamma-camera imaging. The incorporation of other types of agents will enhance MRI or optical imaging. For example, biotinylation of trastuzumab followed by avidin-conjugated to gadolinium-DPTA has been used to enhance MRI detection of breast cancer in experimental models. Other paramagnetic compounds can be coupled to large molecules to facilitate functional MRI imaging of the brain if these coupled compounds are delivered by perispinal extrathecal administration. Optical imaging is particularly useful for imaging of the retina. In this regard, fluorescein-labeling of large molecules delivered by perispinal administration to enable functional imaging of the retina is a method of the present invention. This will allow the investigation of the functional role of the processes mediated by these large molecules in the retina, which will give insight into disease pathogenesis, disease progression, and the effectiveness of treatment.

In addition the methods of the present invention may be used to deliver molecules with a MW less than 2,000 daltons to the brain and other structures of the head more efficiently than if delivered systemically, and these methods utilizing these smaller molecules are also to be considered a part of this invention.

In addition to human use, these methods may be used to image other mammals, including horses, dogs, and cats.

These methods may be used for imaging of humans or other mammals with neurodegenerative diseases, including Alzheimer's Disease, Parkinson's Disease, amyotrophic lateral sclerosis; eye disorders or diseases including, but not limited to, macular degeneration, diabetic retinopathy, sympathetic opthalmia and retinitis pigmentosa; disorders of hearing, including, but not limited to sensorineural hearing loss or presbycusis; central nervous system (CNS) tumors, including tumors of the brain or the spinal cord; other diseases or disorders of the brain, including, but not limited to vascular disorders such as stroke, transient ischemic attack, vascular dementia, and cerebrovascular disease; degenerative disc disease, disc herniation, disc protrusion, disc bulge, sciatica, cervical radiculopathy, and other forms of disc-related pain; tumor metastasis to the spine or spinal cord; low back or neck pain; other diseases or disorders involving the spine, the spinal cord, the spinal nerve roots, the brain, auditory apparatus, or other structures of the head.

The use of cytokine antagonists to treat neurological disorders is the subject of several previous patents of this inventor, including U.S. Pat. Nos. 6,015,557, 6,177,077, 6,419,934 6,419,944, 6,423,321, 6,428,787, 6,537,549, 6,623,736 and US patent applications 20030049256 and U.S. patent application Ser. No. 11/016,047, filed Dec. 18, 2004, entitled “Methods of use of etanercept to improve human cognitive function”, and provisional U.S. patent application 60/585,735, filed Jul. 6, 2004. These issued patents, patent applications, and provisional patent applications are incorporated in their entirety herein. This invention includes further applications of these ideas.

The adverse biologic effects of excess TNF can be reduced by the use of biologic inhibitors of TNF. These inhibitors can be divided into two broad categories: monoclonal antibodies and their derivatives; and TNF binding biologics which are not antibody based. In the first category belong golimumab, also known as CNTO-148 (Centocor, Schering-Plough), infliximab (Remicade®, Centocor), adalimumab (Humira®, Abbott), and CDP 870 (Celltech). The second category includes etanercept, soluble TNF receptor type 1, pegylated soluble TNF receptor type 1 (Amgen) and onercept (Serono). Etanercept has a serum half life of approximately 4.8 days when administered to patients with rheumatoid arthritis on a chronic basis; onercept has a serum half-life which is considerably shorter, and it is usually administered at least three times weekly when used to treat systemic illnesses.

Golimumab has many biologic effects. Golimumab, for example, in addition to being a potent anti-inflammatory also has important anti-apoptotic effects which may be of particular importance in treating neurological disorders, such as certain forms of dementia, where apoptosis plays a pathogenetic role.

Antibodies (immunoglobulins) are proteins produced by one class of lymphocytes (B cells) in response to specific exogenous foreign molecules (antigens). Monoclonal antibodies (mAB), identical immunoglobulin copies which recognize a single antigen, are derived from clones (identical copies) of a single B cell. This technology enables large quantities of an immunoglobulin with a specific target to be mass produced.

Monoclonal antibodies with a high affinity for a specific cytokine will tend to reduce the biologic activity of that cytokine. Substances which reduce the biologic effect of a cytokine can be described in any of the following ways: as a cytokine blocker; as a cytokine inhibitor; or as a cytokine antagonist. In this patent, the terms blocker, inhibitor, and antagonist are used interchangeably with respect to cytokines.

Advances in biotechnology have resulted in improved molecules as compared to simply using monoclonal antibodies. One such molecule is CDP 870 which, rather than being a monoclonal antibody, is a new type of molecule, that being an antibody fragment. By removing part of the antibody structure, the function of this molecule is changed so that it acts differently in the human body. Another new type of molecule, distinct from monoclonal antibodies and soluble receptors, is a fusion protein. One such example is etanercept. This molecule has a distinct function which acts differently in the human body than a simple soluble receptor or receptors.

Monoclonal antibodies, fusion proteins, and all of the specific molecules discussed above under the categories of TNF antagonists and interleukin antagonists are considered biologics, in contrast to drugs that are chemically synthesized. For the purpose of this patent a biologic is defined as a molecule produced through recombinant DNA technology which is derived from the DNA of a living source. The living sources may include humans, other animals, or microorganisms. The biologics mentioned above are manufactured using biotechnology, which usually involves the use of recombinant DNA technology. Cytokine antagonists are one type of biologic. Biologics are regulated through a specific division of the FDA.

Cytokine antagonists can take several forms. They may be monoclonal antibodies (defined above). They may be a monoclonal antibody fragment. They may take the form of a soluble receptor to that cytokine. Soluble receptors freely circulate in the body. When they encounter their target cytokine they bind to it, effectively inactivating the cytokine, since the cytokine is then no longer able to bind with its biologic target in the body. An even more potent antagonist consists of two soluble receptors fused together to a specific portion of an immunoglobulin molecule (Fc fragment). This produces a dimer composed of two soluble receptors which have a high affinity for the target, and a prolonged half-life. This new molecule is called a fusion protein. An example of this new type of molecule, called a fusion protein, is etanercept (Enbrel®).

TNF, a naturally occurring cytokine present in humans and other mammals, plays a key role in the inflammatory response, in the immune response and in the response to infection. TNF is formed by the cleavage of a precursor transmembrane protein, forming soluble molecules which aggregate in vivo to form trimolecular complexes. These complexes then bind to receptors found on a variety of cells. Binding produces an array of pro-inflammatory effects, including release of other pro-inflammatory cytokines, including IL-6, IL-8, and IL-1; release of matrix metalloproteinases; and up regulation of the expression of endothelial adhesion molecules, further amplifying the inflammatory and immune cascade by attracting leukocytes into extravascular tissues.

Golimumab is currently in clinical development by Centocor/Schering-Plough for treatment of rheumatoid arthritis, with potential applications for uveitis, asthma, and Crohn's Disease. It may be described as a immunoglobulin G1, anti-(human tumor necrosis factor α) (human monoclonal CNTO 148 γ1-chain), disulfide with human monoclonal CNTO 148 κ-chain), dimer, and has CAS Registry number 476181-74-5. It is a fully human anti-TNF monoclonal antibody.

Etanercept (Enbrel®, Amgen/Immunex), golimumab, infliximab (Remicade®, Centocor), adalimumab (Humira®, Abbott), CDP 870, and onercept are potent and selective inhibitors of TNF. CDP 870, golimumab and onercept are in clinical development. Etanercept, adalimumab, and infliximab are FDA approved for chronic systemic use to treat rheumatoid arthritis and certain other chronic inflammatory disorders. Golimumab has a molecular weight of approximately 147,000 daltons.

Bevacizumab (Avastin™, Genentech) is a recombinant humanized monoclonal IgG1 antibody that binds to and inhibits the biologic activity of human vascular endothelial growth factor (VEGF) and which may be useful for the treatment of various malignancies. Bevacizumab has a molecular weight of 149,000 daltons and is therefore too large to readily cross the blood-brain barrier if administered systemically.

Etanercept can also be designated as TNFR:Fc because it is a dimeric fusion protein consisting of two soluble TNF receptors fused to a Fc portion of an immunoglobulin molecule. This fusion protein functions in a manner quite distinct from a simple soluble TNF receptor. Soluble TNF receptors are normally present in the human body. But the use of these soluble TNF receptors as imaging agents in this patent is made impractical by their extremely short half-life and therefore their limited biologic activity. The present invention utilizing etanercept is therefore distinguished from an invention specifying the use of a soluble TNF receptor. It is incorrect and imprecise to describe etanercept as a soluble TNF receptor because this is an incorrect description of its complex structure and omits characteristics of etanercept which are absolutely essential to its function. This is further underscored by the developmental history of etanercept. In its first iteration the precursor molecule to etanercept was produced with a single TNF receptor fused to an immunoglobulin fragment. The biologic activity of this molecule was poor. Therefore not only is etanercept distinguished from a soluble TNF receptor, it is also distinguished from a TNF-binding fusion protein which contains the recombinant DNA sequence of only a single soluble TNF receptor. The unique structure of etanercept, containing a dimer (two) soluble TNF receptors fused to an Fc portion of an immunoglobulin molecule, is necessary for the proper performance of the present invention. Since etanercept has the molecular structure of a fusion protein it is thus quite distinct from both onercept, soluble TNF receptor type 1 and pegylated soluble TNF receptor type 1.

The vertebral venous system can also be used to deliver other types of imaging agents to the cerebral cortex, eye, retina, cerebellum, brainstem, eighth cranial nerve, cochlea, inner ear, and cerebrospinal fluid. These imaging agents include pharmacologic agents, other cytokine antagonists, and growth factors which affect neuronal function, or the immune response impacting neuronal function, including, but not limited to: interleukins including IL-1, IL-2, IL-4, IL-6, IL-10, and IL-13; interleukin 1 antagonists, such as IL-1 RA (Kineret®, Amgen) and IL-1 Trap; fusion proteins, such as IL-10 fusion protein and etanercept (Enbrel®, Immunex); human growth hormone and related biologics (recombinant human growth hormone, Humatrope® (somatropin) Eli Lilly & Co., Nutropin®/Nutropin AQ® (somatropin), Geref® (sermorelin) Serono, and Protropin® (somatrem) Genentech)); BDNF; erythropoietin (Epogen® (epoetin alpha) Amgen, Procrit® (epoetin alpha) Johnson & Johnson); G-CSF (Neupogen® (filgrastim), Amgen); GM-CSF; Intron® A (interferon alfa-2b) Schering-Plough; Avonex® (interferon beta-1a) Biogen; bevacizumab (Avastin™, Genentech); pegaptanib, ranibizumab, and other biologic VEGF antagonists; alefacept (LFA-3/lgG1 human fusion protein, Amevive® Biogen); Epidermal growth factor; anti-EGF (ABX-EGF, Abgenix); transforming growth factor-beta 1 (TGF-beta 1); NGF, or other compounds with CNS, vascular or immune imaging activity. Perispinal delivery is particularly advantageous when biologics, such as etanercept, which profoundly affect neuronal function, are administered because of their efficacy at extremely low concentration (high biologic potency).

This method may be used for delivery for humans or other mammals with neurodegenerative diseases, including Alzheimer's Disease, Parkinson's Disease, amyotrophic lateral sclerosis; for eye disorders or diseases including, but not limited to, macular degeneration, diabetic retinopathy, sympathetic opthalmia and retinitis pigmentosa; disorders of hearing, including, but not limited to sensorineural hearing loss or presbycusis; central nervous system (CNS) tumors, including tumors of the brain; for other diseases or disorders of the brain, including, but not limited to vascular disorders such as stroke, transient ischemic attack, vascular dementia, and cerebrovascular disease; infectious diseases of the CNS, including viral and bacterial infections; for sciatica, cervical radiculopathy, and other forms of disc-related pain; for low back pain; other diseases or disorders involving the spine, the spinal cord, the spinal nerve roots, the brain, eyes, auditory apparatus, or other structures of the head.

Localized administration for the treatment of localized clinical disorders has many clinical advantages over the use of conventional systemic treatment. Locally administered medication after delivery diffuses through local capillary, venous, arterial, and lymphatic action to reach the imaging target. In addition local administration of a large molecule, such as goliumumab, defined as a molecule with a molecular weight greater than or equal to 2,000 daltons, in the vicinity of the spine (perispinal administration) without direct intrathecal injection has the key advantage of improved delivery of the molecule to the brain and across the blood-brain barrier (BBB), with delivery enhanced by transport via the vertebral venous system. Intrathecal injection delivers the molecule into the cerebrospinal fluid (CSF), but has disadvantages of possible infection, hemorrhage, and subsequent CSF leak.

The BBB is a physiologic barrier which separates the brain and cerebrospinal fluid from the blood. It consists of a layer of cells which comprise the cerebral capillary endothelium, the choroid plexus epithelium, and the arachnoid membranes, which are connected by tight junctions (zonulae occludens). These tight junctions may be as much as 100 times tighter than junctions of other capillary endothelium, and prevent molecules larger than about 600 daltons in molecular weight (MW) from traversing the BBB when the molecule is administered systemically i.e. by conventional subcutaneous, intramuscular, or intravenous injection at an anatomic site remote from the spine.

The vertebral venous system (VVS) is an interconnected plexus of veins which surrounds the spinal cord and extends the entire length of the spine. This venous system provides a vascular route from the pelvis to the cranium which richly involves the bone marrow of the spine and which is functionally distinct from the systemic venous system. First described by Willis in 1663, the functional significance of the vertebral venous system was largely unappreciated until the work of Batson, who in 1940 proposed that this venous plexus provided the route by which prostate cancer metastasizes to the vertebral column. Acceptance of Batson's proposal by the medical community has led to the designation of the vertebral venous system as Batson's Plexus. Although now widely appreciated as a possible route by which cancer cells may spread to the spine there have been no previous suggestions that Batson's plexus may be of diagnostic usefulness. The use of Batson's plexus as route of delivery of biologics for clinical use, and as a route for delivery of large molecules to the brain, the eye, the retina, the auditory apparatus, the cranial nerves or the head are inventions of the author. This patent is a continuation to the methods of use of Batson's plexus to deliver therapeutic molecules to the nervous system which has been previously proposed by the inventor, and incorporates the inventor's previous patents and patent applications discussing this.

Perispinal administration involves anatomically localized delivery performed so as to place the diagnostic molecule directly in the vicinity of the spine at the time of initial administration. For the purposes of this patent, “in the vicinity of” is defined as within 10 centimeters. Perispinal administration includes, but is not limited to, the following types of administration: parenteral; subcutaneous; intramuscular; or interspinous; and specifically includes the use of interspinous injection carried through the skin in the midline of the neck or back, directly-overlying the spine. For the purposes of this patent perispinal administration excludes intrathecal administration, which carries additional risks of infection and hemorrhage. Therefore in this patent “perispinal” is more exactly defined as “perispinal (extrathecal)”, but for the purposes of brevity shall be designated throughout simply as “perispinal”. Perispinal administration leads to enhanced delivery of large molecules to the brain and the head and the structures therein in a diagnostically effective amount. The conventional mode of delivery of these molecules for clinical applications, i.e. subcutaneous administration in the abdomen, thighs, or arms, does not effectuate delivery across the blood-brain barrier (see Robinson reference 60) which is as efficient as perispinal administration and is therefore distinguished from the perispinal methods of administration described in this invention.

### 4. DESCRIPTION OF THE PRIOR ART

Pharmacologic chemical substances, compounds and agents having various organic structures and metabolic functions which are used for the treatment of sensorineural hearing loss, and TNF related diseases have been disclosed in the prior art. One example is U.S. Pat. No. 5,837,681, entitled “Method For Treating Sensorineural Hearing Loss Using Glial Cell Line-Derived Neurotrophic Factor (GDNF) Protein Product”. However, this prior art patent does not teach the use of a TNF antagonist delivered via the vertebral venous system, as in the present invention, and GDNF has biologic actions which are clearly distinct from those of the TNF binding biologics of the present invention.

U.S. Pat. No. 6,043,221 entitled “Method For Preventing And Treating Hearing Loss Using A Neuturin Protein Product” discusses the use of a neurotrophic factor. This prior art patent does not teach the use of a TNF antagonist delivered via the vertebral venous system to image disorders of the brain, as in the present invention.

U.S. Pat. No. 5,385,901 entitled “Method Of Treating Abnormal Concentrations of TNF Alpha” discloses a method for the use of TNF antagonists. This prior art patent does not teach the use of a biologic delivered via the vertebral venous system as described in the present invention for the suppression and inhibition of the action of TNF in the human body to image disorders of the brain, as in the present invention.

U.S. Pat. No. 5,434,170 entitled “Method For Treating Neurocognitive Disorders” discloses the use of thalidomide to treat dementia. This prior art patent does not teach the use of etanercept or another biologic delivered via the vertebral venous system as described in the present invention to image disorders of the brain.

U.S. Pat. No. 6,277,969 discloses the use of anti-TNF antibodies for treatment of various disorders. This prior art patent does not teach the use of etanercept or another biologic delivered via the vertebral venous system as described in the present invention to image disorders of the brain.

U.S. Patent application 2004/0258671 by Watkins entitled “Methods for Treating Pain” discloses the use of IL-10 and IL-10 fusion protein and other biologics for treating pain. This patient application does not disclose the use of these substances to image disorders of the brain.

U.S. Pat. No. 5,656,272 to LE et. al. discloses the use of TNF inhibitors for treatment of various disorders, including the use of anti-TNF monoclonal antibodies. This prior art patent does not teach the use of etanercept or another biologic delivered via the vertebral venous system as described in the present invention to image disorders of the brain.

U.S. Pat. No. 5,650,396 discloses a method of treating multiple sclerosis (MS) by blocking and inhibiting the action of TNF in a patient. This prior art patent does not teach the use of etanercept or another biologic delivered via the vertebral venous system as described in the present invention to image disorders of the brain.

U.S. Pat. No. 5,605,690 discloses the use of TNF inhibitors for treatment of various disorders. This prior art patent does not teach the use of etanercept or another biologic delivered via the vertebral venous system as described in the present invention to image disorders of the brain.

U.S. patent application US 2003/0148955 to Pluenneke discloses the use of biologic TNF inhibitors, including etanercept, for the treatment of medical disorders. However, it does not give an enabling disclosure of the use of etanercept for the imaging of disorders of the brain utilizing the vertebral venous system as does the present invention and it does not predate the U.S. Pat. No. 6,015,557 of the present inventor of which this patent application is a continuation-in-part.

U.S. Pat. Nos. 7,115,557, 6,649,589 and 6,635,250 and related patent applications which have not been granted, to Olmarker and Rydevik, and previous publications by Olmarker (see References) discuss the use of TNF inhibitors for the treatment of nerve root injury and related disorders. These patents do not teach the use of etanercept or another biologic delivered via the vertebral venous system as described in the present invention to image disorders of the brain, and are not enabling with respect to etanercept, golimumab, certolizumab pegol, and other molecules discussed herein.

U.S. Pat. No. 5,863,769 discloses using IL-1 RA for treating various diseases. This prior art patent does not teach the use of an interleukin antagonist or other biologic delivered via the vertebral venous system as described in the present invention to image disorders of the brain.

U.S. Pat. No. 6,013,253 discloses using interferon and IL-1 RA for treating multiple sclerosis. This prior art patent does not teach the use of an interleukin antagonist or other biologic delivered via the vertebral venous system as described in the present invention to image disorders of the brain.

U.S. Pat. No. 5,075,222 discloses the use of IL-1 inhibitors for treatment of various disorders. This prior art patent does not teach the use of an interleukin antagonist or other biologic delivered via the vertebral venous system as described in the present invention to image disorders of the brain.

U.S. Pat. No. 6,159,460 discloses the use of IL-1 inhibitors for treatment of various disorders. This prior art patent does not teach the use of an interleukin antagonist or other biologic delivered via the vertebral venous system as described in the present invention to image disorders of the brain.

U.S. Pat. No. 6,096,728 discloses the use of IL-1 inhibitors for treatment of various disorders. This prior art patent does not teach the use of an interleukin antagonist or other biologic delivered via the vertebral venous system as described in the present invention to image disorders of the brain.

U.S. Pat. No. 6,548,527 to Rahman discloses the use of etanercept for the treatment of immune mediated ear disorders. This prior art patent does not teach the use of etanercept or other biologic delivered via the vertebral venous system as described in the present invention to image disorders of the brain.

US patent application 20040072885 to Rahman discloses the use of etanercept for the treatment of immune mediated ear disorders. This prior art patent does not teach the use of an etanercept or other biologic delivered via the vertebral venous system as described in the present invention to image disorders of the brain.

An article (Rahman M U, Poe D S, Choi H K. Etanercept therapy for immune-mediated cochleovestibular disorders: preliminary results in a pilot study. Otol Neurotol. 2001 September; 22(5):619-24.) disclosed the use of etanercept by subcutaneous administration for the treatment of immune mediated ear disorders. This prior art patent does not teach the use of etanercept or other biologic delivered via the vertebral venous system as described in the present invention to image disorders of the brain. Clemens (reference 57) demonstrated that the internal and external vertebral venous plexuses freely intercommunicate, and this was also demonstrated by Vogelsang (reference 58) with the use of intraosseous spinal venography. But neither Clemens nor Vogelsang discussed the use of the VVS to facilitate delivery of large molecules to the brain.

Groen (reference 50) confirmed the fact that all three divisions of the vertebral venous system (internal and external plexuses, and the basivertebral veins) freely intercommunicated, and that all divisions of this system lacked valves. But Groen did not discuss the use of the VVS to facilitate delivery of large molecules to the brain.

Two recent articles (Lirk references 54 and 55) discuss an anatomic finding, disclosing the existence of a gap in a ligamentous barrier to the epidural space. These articles, however, do not discuss the administration of large molecules by the perispinal route, or the relevance of this anatomic finding to the delivery of large molecules to the brain.

Batson in 1940 (reference 47) published information regarding the vertebral venous system. Experimentally he demonstrated a connection between the pelvic venous system and the vertebral venous system, and proposed that this was a route whereby carcinoma originating in the pelvis could metastasize to the spine. His work did not disclose the methods of the present invention for delivery of large molecules to the brain.

Ruiz and Gisolf (references 44 and 45) have recently published articles discussing the vertebral venous system and its connections to the cranial venous system. Neither authors discuss the potential use of this system as a route of administration of large molecules to the brain.

Retrograde cerebral perfusion has been previously demonstrated to deliver dye to the surface of the brain in pigs after superior vena caval injection (Ye reference 42)) but the authors did not propose the use of this route to deliver large molecules to the brain.

Several authors (references 44-50) have discussed the anatomy and function of the vertebral venous system but none have proposed the use of the vertebral venous system as a route of delivery of large molecules to the brain, nor have they proposed the methods of the present invention.

Two articles by Byrod discussed a mechanism whereby substances applied epidurally can cross into the endoneurial space (Byrod references 51 and 52), but neither article discusses the perispinal use of a large molecule for delivery to the brain.

Robinson (reference 60) states the prevailing view that systemic administration of etanercept does not lead to therapeutic concentrations of etanercept in the brain, because systemically administered etanercept does not cross the BBB.

Markomichelakis (reference 62) in 2005, following the issuance of U.S. Pat. No. 6,428,787 by this inventor which claimed the use of infliximab to treat macular degeneration, described the regression of macular degeneration following infliximab treatment given systemically. This reference did not describe or discuss the use of perispinal infliximab.

None of the prior art patents disclose or teach the use of perispinal administration of large molecules as in the present invention as a way of delivering large molecules to the brain, the eyes, or the head, in which this method of administration provides a method of improved imaging for diagnostic purposes utilizing these large molecules.

Accordingly, it is an object of the present invention to provide various large molecule imaging agents administered through the perispinal route as a new method for diagnostic imaging.

Another object of the present invention to provide various large molecule imaging agents delivered by persipinal extrathecal administration as a new method for gauging the effectiveness of treatment.

Another object of the present invention to provide various large molecule imaging agents delivered by persipinal extrathecal administration as a new method for gauging disease progression.

Another object of the present invention to provide various large molecule imaging agents delivered by persipinal extrathecal administration for gauging progression of Alzheimer's disease and other forms of dementia over time.

Another object of the present invention to provide radiolabeled large molecules delivered by persipinal extrathecal administration to facilitate or enable imaging of amyloid in the brain.

Another object of the present invention to provide radiolabeled imaging agents delivered by persipinal extrathecal administration for imaging of the brain.

Another object of the present invention to provide radiolabeled imaging agents delivered by persipinal extrathecal administration as a new method to enhance or enable functional imaging of the brain.

Another object of the present invention to provide radiolabeled imaging agents delivered by persipinal extrathecal administration for imaging of the spine and/or spinal cord.

Another object of the present invention to provide radiolabeled imaging agents delivered by persipinal extrathecal administration as a new method to enhance or enable functional imaging of the spine, intervertebral discs, or spinal cord as an aid in the diagnosis of back or neck pain.

### 5. SUMMARY OF THE INVENTION

The present invention provides specific methods for delivering large molecules to a mammal utilizing perispinal administration without direct intrathecal injection for diagnostic purposes. For the purposes of this patent “perispinal” is to be considered as referring to “perispinal extrathecal”; therefore direct intrathecal administration is excluded from the methods discussed.

As used herein, “diagnostically effective” refers to the material or amount of material which is effective to help diagnose one or more symptoms or signs of a disease or medical condition, or help to measure or quantify disease progression or the effect of treatment in a mammal.

As used herein, “subject” refers to animals, including mammals, such as human beings, domesticated animals, and animals of commercial value.

As used herein, the term “biologic” is defined as a drug which is derived or prepared from the DNA of a living organism, which has a relatively large molecular weight and a high structural complexity as compared with biologically active substances which are produced by chemical synthesis. The living sources from which biologics may be obtained include humans, other animals, and microorganisms. The drug may be produced by recombinant means, or may be extracted and purified directly from the living source.

As used herein, “perispinal administration without direct intrathecal injection” refers to administration adjacent to the spine, but outside of the intrathecal space (extrathecal), wherein the injection needle or catheter does not penetrate the dural barrier. Administration therefore is not directly into the cerebrospinal fluid.

Non-brain capillaries are made up of endothelial cells which are separated by small gaps that allow chemicals in solution to pass into the blood stream, where they can be transported throughout the body. In non-brain capillaries, compounds having molecular weights greater than 25,000 Daltons can undergo transport. In contrast, endothelial cells in brain capillaries are more tightly packed, due to the existence of zonula occludentes (tight junctions) between them, thereby blocking the passage of most molecules. The blood-brain barrier blocks most molecules except those that cross cell membranes by means of lipid solubility (such as, for example, oxygen, carbon dioxide, and ethanol) and those which are allowed in by specific transport systems (such as, for example, sugars, amino acids, purines, nucleosides and organic acids). Generally, it is accepted that substances having a molecular weight greater than 500 daltons cannot cross the blood-brain barrier, whereas substances having a molecular weight less than 500 daltons can cross the blood-brain barrier.

Because they do not effectively cross the blood-brain barrier, biologics having a molecular weight greater than 500 are not effective for imaging the brain when administered systemically. For example, etanercept has a molecular weight of 150,000 Daltons, and is not effective for imaging conditions of the brain, eye, spine, spinal cord, or cranial nerves when delivered systemically. Thus, utilization of the VVS is particularly useful for the administration of high molecular weight biologics such as bevacizumab or etanercept, for delivery to the brain, retina, eye, cranial nerves, spine and spinal cord, thereby enabling imaging of a wide range of disorders of the brain, the retina, and the nervous system, including those which are inflammatory, malignant, infectious, autoimmune, vascular, and degenerative.

In addition the methods of the present invention may be used to deliver molecules with a MW less than 2,000 daltons to the brain and other structures of the head more efficiently than if delivered systemically, and these methods utilizing these smaller molecules are also to be considered a part of this invention.

Perispinal administration involves anatomically localized delivery performed so as to place radiolabeled etanercept or another tagged biologic directly in the vicinity of the spine, and thereby facilitate delivery of the large molecule to the brain, the eye, the retina, the auditory apparatus, the cranial nerves, the spinal nerve roots, the intervertebral discs, the spinal nerve roots, the dorsal root ganglia, the spinal cord or the head. Perispinal administration includes, but is not limited to, the following types of administration: parenteral; subcutaneous; intramuscular; and interspinous; and specifically includes the use of interspinous injection carried through the skin in the midline of the neck or back, directly overlying the spine, so that the large molecule is delivered into the interspinous space. Perispinal administration leads to enhanced delivery of the imaging molecule to the brain, the eye, the retina, the auditory apparatus, the spine and contiguous structures, and the cranial nerves or the head in an amount effective to facilitate imaging, via the vertebral venous system. Delivery of a large molecule to the brain utilizing the methods of the present invention includes the use of the vertebral venous system to deliver the large molecule to the brain via retrograde venous flow. Physical positioning may also be used to enhance delivery via this route.

All of the large molecules available for therapeutic use are approved for systemic administration, either by subcutaneous (SC) or intravenous (IV) administration. None have been approved for perispinal or interspinous administration.

This patent application describes novel methods of administration of large molecules, utilizing perispinal administration, which results in improved imaging efficiency (decreased dose for equivalent diagnostic effect) and/or increased effectiveness (increased diagnostic effect for equivalent therapeutic dose) compared with systemic administration.

The same methods described for etanercept of this invention also apply to other large molecules, including, but not limited to, golimumab, certolizumab pegol, IL-1 Trap, Kineret®, bevacizumab, pegaptanib, ranibizumab, rituximab, Zevalin®, Mylotarg®, Campath®, HumaSpect®, abatacept, cetuximab, panitumumab, pegfilgrastim, filgrastim, erythropoietin, Aranesp®, trastuzumab, Pegasys®, Intron A®, PEG-Intron®, Infergen®, Avonex®, Rebif®, Betaseron®, Actimmune®, Ontak®, Simulect®, Zenapax®, Genkaxin®, recombinant human growth hormone, reteplase, alteplase, tPA (tissue plasminogen activator), urokinase plasminogen activator, streptokinase, urokinase, transforming growth factor-beta, immune globulin, anti-amyloid antibodies, anti-tau antibodies, AAB-001, AAB-002, and smaller molecules, such as Tarceva®, all of which maybe given by perispinal administration, and whose use, when radiolabeled or unaltered, by perispinal administration without direct intrathecal injection, for either diagnosis or treatment, constitute part of this invention.

### 6. DETAILED DESCRIPTION OF THE PREFERRED EMBODIMENTS

The use of perispinal administration of cytokine antagonists to treat neurological disorders is discussed in US patent application 20030049256 of this inventor. The use of perispinal administration without direct intrathecal injection and the vertebral venous system to deliver large molecules to the brain, the eye, and the auditory apparatus are discussed in the following provisional patent applications:

60/585,735 filed Jul. 6, 2004;
60/659,414 filed Mar. 9, 2005;
60/662,744 filed Mar. 17, 2005;
and 60/669,022, filed Apr. 7, 2005,

Perispinal administration of a molecule when compared to systemic administration, carries with it one or more of the following advantages for the present invention:
1) greatly improved efficacy due to improved delivery of the diagnostic molecule to the brain, the eye, the retina, the auditory apparatus, the cranial nerves, the spinal nerve roots, the dorsal root ganglia, the spinal cord or the head via the vertebral venous system (VVS).
2) greater efficacy due to the achievement of higher local concentration in the interspinous space, leading to improved delivery to the VVS and the brain, the eye, the retina, the auditory apparatus, the cranial nerves, the spinal nerve roots, the dorsal root ganglia, the spinal cord or the head.
3) greater efficacy due to the ability of the administered diagnostic molecule to reach the brain, the eye, the retina, the auditory apparatus, the cranial nerves, the spinal nerve roots, the dorsal root ganglia, the spinal cord or the head without degradation caused by hepatic or systemic circulation;
4) more rapid onset of action;
5) longer duration of action; and
6) Potentially fewer side effects, due to lower required dosage.

These advantages apply to both large molecules, such as monoclonal antibodies, which typically have a MW of more than 100,000 daltons, and to smaller molecules, many of which, even though they have a MW less than 2,000 daltons, have difficulty traversing the BBB. Even smaller molecules, those with a MW less than 500 daltons, which often can cross the BBB, will achieve a greater concentration in brain or eye tissue if administered by perispinal delivery without direct intrathecal injection, especially if immediately following injection the postural adjustments are made to direct the head downward with the body in a Trendelenburg position, thereby facilitating retrograde venous perfusion via the intracranial anastomoses of the vertebral venous system. The blood-eye barrier, for the purposes of this patent, will be traversed by the methods of the present invention in a manner equivalent to the manner in which these molecules cross the blood-brain barrier. The blood-nerve barrier protecting the spinal nerve roots and the spinal cord, consisting in large part of the barrier formed by the dura mater, will also be traversed in a manner utilizing the methods of the present invention i.e. by carriage in the vertebral venous system, etc.

The inventor has extensive clinical experience utilizing perispinal injection of etanercept for the treatment of disc-related pain and radiculopathy, including low back pain, neck pain, lumbar radiculopathy (sciatica), cervical radiculopathy, pain associated with annular tear of the intervertebral disc, and pain associated with degenerative disc disease (see Tobinick reference 63) (Tobinick, E. and S. Davoodifar, Efficacy of etanercept delivered by perispinal administration for chronic back and/or neck disc-related pain: a study of clinical observations in 143 patients. Curr Med Res Opin, 2004. 20(7): p. 1075-85). In this article the inventor reported the results of perispinal etanercept treatment for 143 patients, including those with disc bulge, protrusion, extrusion or herniation; lumbar and cervical radiculopathy; degenerative disc disease; central spinal stenosis; spondylolisthesis; back pain, neck pain, or sciatica; and annular tear of the intervertebral disc. The 143 patients had a mean duration of pain of 9.8 years. After a mean of 2.3 doses of perispinal etanercept the mean VAS intensity of pain, sensory disturbance, and weakness was significantly reduced at 20 min., 1 day, 1 week, 2 weeks, and 1 month. In a previous publication (Tobinick, E. L. and S. Britschgi-Davoodifar, Perispinal TNF-alpha inhibition for discogenic pain. Swiss Med Wkly, 2003. 133(11-12): p. 170-7) the inventor documented clinical improvement following perispinal etanercept in a cohort of 20 patients with the following diagnoses: acute lumbar radiculopathy; chronic cervical and lumbar discogenic pain; subacute lumbar radiculopathy; chronic discogenic pain and failed back surgery syndrome; chronic low back pain and sciatica; chronic, treatment-resistant discogenic pain. Rapid, substantial, and sustained clinical pain reduction and improvement in functional disability was documented in this group of patients for a mean of 230 days. The vertebral venous system drains the perispinal area, including both the deeper interspinous space superficial to the ligamentum flavum and the subcutaneous perispinal space which overlies the spinous processes and the deeper interspinous space. It is a method of the present invention to introduce large molecules into this area (the perispinal area) to enable them to drain into the vertebral venous system and thereby cross the blood-nerve barrier and produce diagnostic benefit for imaging the spinal conditions enumerated in this paragraph. This may be accomplished by perispinal injection of these molecules, which leads to entry of the large molecules into the vertebral venous system, and then delivery of these molecules, by retrograde venous flow due to lack of venous valves in the VVS, to the spinal nerve roots, the dorsal root ganglia, and the spinal cord. In the case of etanercept and golimumab, for example, this results in neutralization of excess TNF and clinical improvement in patients suffering from a variety of spinal ailments, including specifically those enumerated in this paragraph. Doses smaller than the therapeutic dose of these molecules may be used if the molecules are radiolabeled and used for diagnostic imaging. Perispinal administration of these molecules when radiolabeled or otherwise tagged so they can be identified upon imaging will indicate the anatomic source of excess TNF-alpha, thereby improving diagnosis.

For example, in a patient with known degenerative disc disease involving multiple intervertebral discs a frequent problem is the identification of the active “pain generator”. This is a common problem, because patients with multiple abnormal discs are common, and are often considered for disc replacement or spinal fusion. In these surgical cases it is essential to identify the source of the patient's pain prior to surgery, because failure to replace the proper disc could lead to failure to alleviate the patient's severe spinal pain. Currently, however, there is no biologic marker to indicate which disc is responsible for the pain. Improved imaging, utilizing a radiolabeled or otherwise tagged cytokine antagonist delivered by perispinal administration helps to improve the accuracy of this identification, thereby improving treatment response. In the case of tagged etanercept one may also expect a therapeutic effect. Therefore the use of a tagged biologic TNF antagonist, such as etanercept, would result in both an improved therapeutic effect and improved diagnosis.

The inventor has successful clinical experience with perispinal administration of etanercept, a large molecule (MW 149,000 daltons) for the treatment of Alzheimer's Disease (AD) (see experimental results infra) which illustrates the clinical efficacy of this method of delivery of large molecules for the treatment of brain disorders, and, specifically, the ability of this delivery method to enable etanercept to cross the BBB and effectively treat AD. It should be noted that a previous clinical trial utilizing etanercept (reference 61) delivered systemically (by subcutaneous administration remote from the spine) failed to show efficacy, thereby providing prima facie evidence of the superiority of perispinal administration to deliver etanercept to the brain, when a comparison of the failed trial results to the successful experimental results obtained utilizing perispinal administration of etanercept, detailed infra, is made. The methods described herein involve the use of perispinal administration to effectively deliver large molecules to the brain, the eye, the retina, the auditory apparatus, the cranial nerves or the head, for diagnostic use in humans and other mammals.

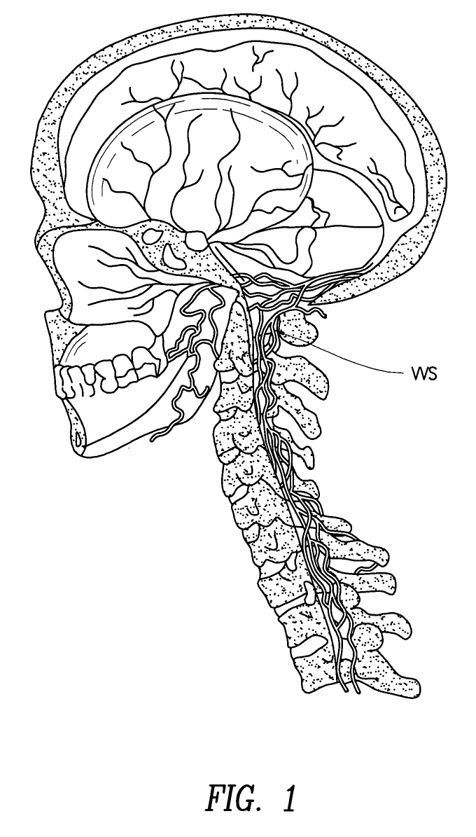

The VVS consists of an interconnected and richly anastomosed system of veins which run along the entire length of the vertebral canal. The vertebral venous plexus, for descriptive purposes, has been separated into three intercommunicating divisions: the internal vertebral venous plexuses (anterior and posterior) lying within the spinal canal, but external to the dura; the external vertebral venous plexuses (anterior and posterior) which surround the vertebral column; and the basivertebral veins which run horizontally within the vertebrae (see FIG. 1, drawn by Frank Netter, MD, which follows the text portion of this patent application and is included as an integral part of the application). Both the internal and external vertebral venous plexus course longitudinally along the entire length of the spine, from the sacrum to the cranial vault. Utilizing corrosion casting and injections of Araldite, Clemens demonstrated that the internal and external vertebral venous plexuses freely intercommunicate, and this was also demonstrated by Vogelsang with the use of intraosseous spinal venography. Groen and his colleagues with an improved Araldite injection technique which utilized thrombolytics, confirmed the fact that all three divisions of the vertebral venous system (internal and external plexuses, and the basivertebral veins) freely intercommunicated, and that all divisions of this system lacked valves. The internal vertebral venous plexus communicates with the intraspinal and radicular veins and freely communicates with the external vertebral venous plexus via the intervertebral veins (see references 44-50). In addition, the VVS communicates with the azygous veins, and has other connections to the caval venous system, but not efficiently. Therefore a conventional intravenous injection in the antecubital fossa, for example, or into one of the large veins of the forearm, which delivers a solution containing a given therapeutic molecule into the caval venous system, does not efficiently deliver the same therapeutic molecule to the VVS. Likewise, delivery of a solution containing a given therapeutic molecule by perispinal administration will not result in efficient delivery of the given therapeutic molecule into the caval venous system, but will result in efficient delivery into the VVS. The caval venous system and the VVS are separate and largely independent (see reference 59), although they are interconnected, although not in an efficient manner. To phrase the same thoughts in a different way, it would be accurate to say that perispinal administration of a large molecule will result in efficient delivery of the large molecule to the VVS, with only a small amount of delivery of the large molecule into the caval venous system. Delivery of the same large molecule by intravenous infusion into an arm vein, for example, will deliver the large molecule to the caval venous system, expose the large molecule to dilution throughout the body, and fail to deliver the large molecule to the brain, the eye, the retina, the auditory apparatus, the cranial nerves or the head.

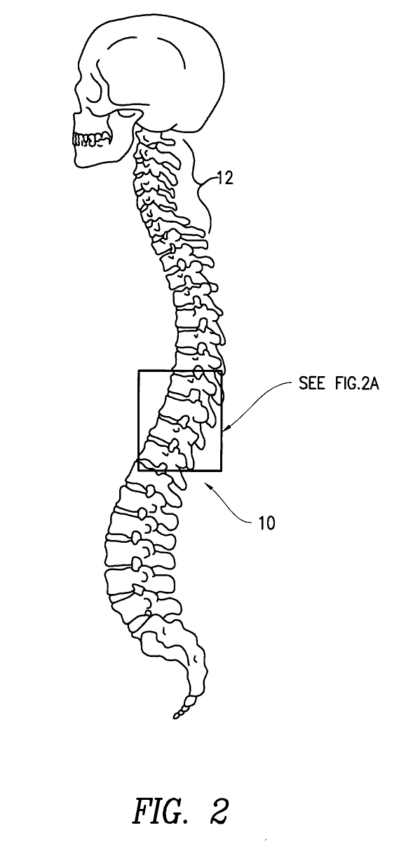

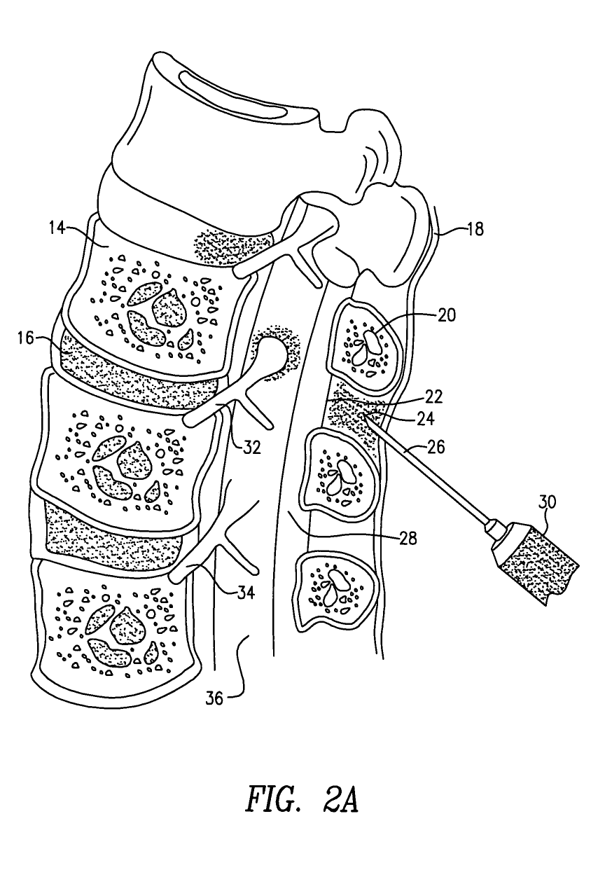

A specific anatomic route, by which a large molecule delivered by perispinal administration reaches the brain, has been defined by the inventor (see FIG. 2). This route is as follows. A large molecule is delivered to the interspinous space in proximity to the ligamentum flavum by percutaneous injection through the skin by midline interspinous needle injection. Large molecules delivered to the interspinous space in this way (being the anatomic region in the midline of the back, in-between two adjacent spinous processes) are delivered into the VVS because the VVS serves to provide venous drainage to the interspinous space and subcutaneous space which is posterior to the spine (see Batson references 48 and 49 for a discussion of the VVS, which, however, does not discuss the therapeutic potential of the VVS). Solutions injected into this area, therefore, will be preferentially absorbed into the VVS rather than into the caval venous system. In addition, a more direct route to the epidural space is also possible for solutions injected into the interspinous space, by travel through midline defects in the ligamentum flavum. Midline defects in the ligamentum flavum are common, particularly in the cervical region. When present the midline ligamentum flavum defect provides a direct route of access for large molecules to the epidural space. Within the epidural space lies a richly interconnected venous plexus (which is part of the VVS), which is valveless and which is capable of transporting large molecules rapidly in the cephalad or caudad directions (see Batson references 48 and 49). Flow within the VVS is bidirectional. Therefore large molecules injected into the interspinous space drain directly into the VVS and thereby gain direct access to the brain, if the patient is positioned properly immediately following injection so that gravity is used to direct flow via the VVS toward the brain. This is possible because the flow within the VVS can be bidirectional; therefore these veins serve not only to drain blood from the brain, but also to deliver venous blood to the brain, in retrograde fashion, via the venous connections of the VVS with the intracranial venous system, including the dural sinuses. This retrograde flow is made possible by the lack of venous valves in the VVS. Retrograde venous delivery of large molecules to the brain is a method of the present invention and a discovery of the inventor. The author has detailed much of his current thinking regarding the vertebral venous system and its connection with the cerebral venous system in a recently published article entitled “The Cerebrospinal Venous System Anatomy, Physiology, and Clinical Implications” published in Medscape General Medicine in February 2006 (Med Gen Med. 2006 Feb. 22; 8(1):53.) This article is incorporated in its entirety in this patent application by reference.

The VVS can be used to deliver large biologic diagnostic agents (i.e., biologics having a molecular weight greater than 600 Daltons, preferably greater than 2000 Daltons) utilizing retrograde venous flow from the VVS into the cranial venous sinuses and the intracranial venous system for delivery to the cerebral cortex, eye, retina, spine, cerebellum, brainstem, eighth cranial nerve, cochlea, inner ear, cerebrospinal fluid, spine, spinal cord, dorsal root ganglion, spinal nerve roots, reproductive organs and spinal nerve roots of a subject. Exemplary pharmaceutically acceptable diagnostic agents may include pharmacologic agents, cytokine antagonists and growth factors which can affect neuronal function or the immune response impacting neuronal function, including, but not limited to, for example, golimumab, CDP 870, and etanercept.

Retrograde venous delivery of large molecules to the brain is facilitated by body positioning after interspinous injection. For example, if following cervical interspinous injection the patient is placed in the head down trendelenburg position then the inventor has discovered that this will lead to effective delivery of the large molecule to the brain, via retrograde flow in the VVS into the cranial venous system.

### BRIEF DESCRIPTION OF THE FIGURES

### See U.S. patent application Ser. No. 11/016/047 Filed by the Inventor on Dec. 18, 2004 (Incorporated Herein by Reference) for Access to the Figures Referenced Here

FIG. 1 is a scan of a photograph, taken at the National Library of Medicine, of plate 5 drawn by Breschet and published in 1828 (reference 56), depicting the cranial and vertebral venous systems, their anastomoses, and their anatomic characteristics, especially in relationship to other anatomic features of the brain and spine.

FIG. 2 is a diagram depicting perispinal administration, in accordance with the present invention.

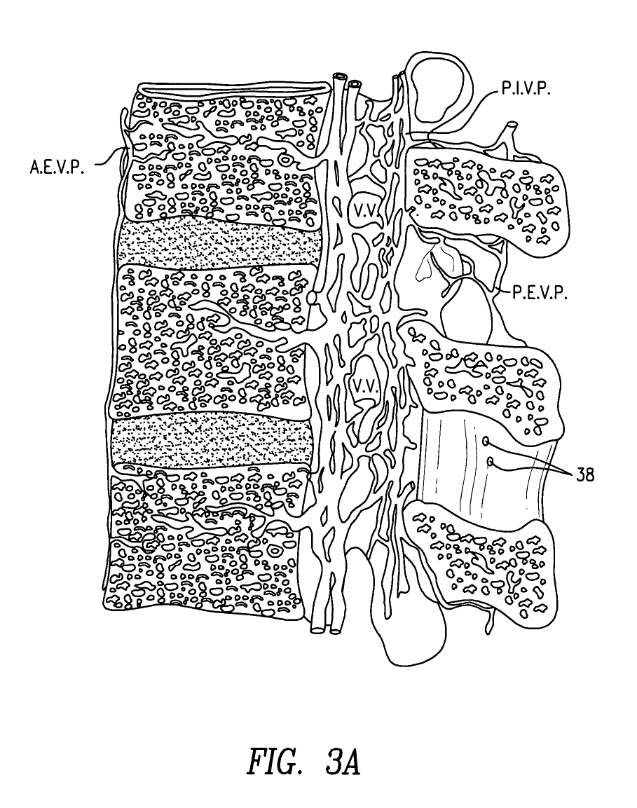

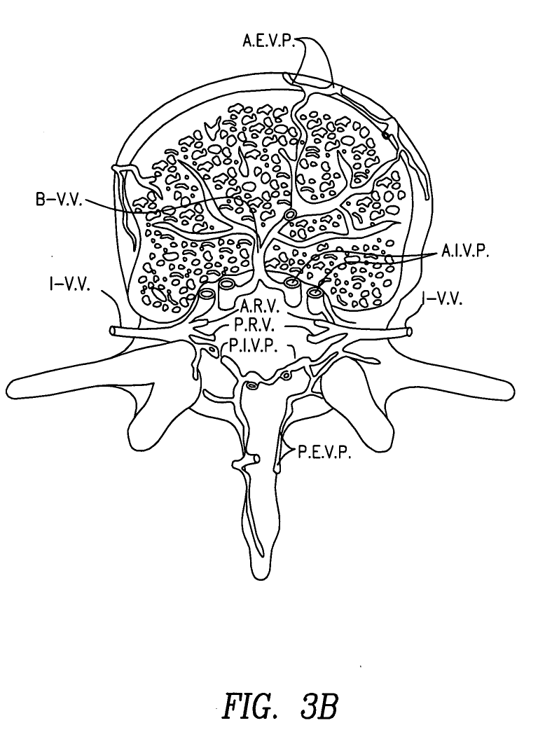

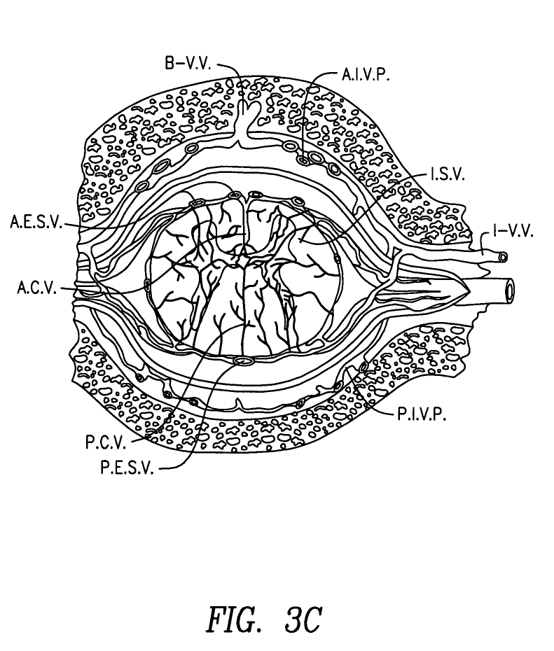

FIG. 3 are drawings by Frank Netter, MD depicting three different anatomic views of the vertebral venous system (VVS) and its anatomic relationship to the interspinous space and other anatomic elements of the spine.

FIG. 1 depicts the anastomoses between the cranial and vertebral venous systems. Perispinal administration for delivery to the brain and other structures of the head is preferably performed by a percutaneous injection into an interspinous space in the posterior cervical area (12 in FIG. 2). As shown in more detail in FIG. 2, hollow needle (26) containing etanercept (or other diagnostic molecule of this invention) in solution (30) is injected through the skin 18 into the interspinous space 24. If the needle were carried further it could penetrate the ligamentum flavum (22), delivering the diagnostic molecule into the epidural space (28) surrounding the spinal cord (36), although in most iterations of this invention the ligamentum flavum is not penetrated by the needle, and the diagnostic molecule is deposited into the interspinous space more superficially, without penetration of the ligamentum flavum. The diagnostic molecule in the interspinous space drains into the vertebral venous system, and is then carried to the brain, the eye, the auditory apparatus, and other structures of the head. (34) is a spinal nerve root.

The interspinous space (24) is defined as the space between two adjacent spinous processes (20). FIG. 3 shows the interspinous space (24) having veins (38) (FIG. 3) which collect the diagnostic molecule, in this case etanercept, which reaches the interspinous space after percutaneous interspinous injection and which veins drain said diagnostic molecule into the VVS, so that, utilizing the physical maneuvers of the present invention, the diagnostic molecule is transported via retrograde venous flow into the intracranial veins via the anastomoses depicted in FIG. 1, and thence to the brain, the eye, the auditory apparatus, or other structures of the head.

The inventor is using the vertebral venous system in a non-obvious way for the inventions disclosed herein. For a venous system is routinely conceptualized as a system that drains blood from a target area or organ. For example the venous system which drains the kidneys is widely acknowledged to be a vascular system that drains blood from the kidneys, not as a way of delivering a therapeutic molecule to the kidneys. Likewise the venous system of the brain is widely medically recognized as a system which functions to drain blood from the brain. It would be counter-intuitive to propose using the VVS to deliver a diagnostic molecule to the brain, by conventional thinking. Likewise the use of the vertebral venous system to achieve delivery of diagnostic compounds to the brain is not obvious, because conventional thinking is that this venous system functions to drain venous blood away from these anatomic sites. Therefore the inventions of consideration here are in this way counter-intuitive, because they rely on the vertebral venous system to deliver diagnostic molecules (including specifically large molecules) to the brain, the eye, the retina, the auditory apparatus, the cranial nerves or the head. This delivery is accomplished by retrograde venous flow (opposite from the usual direction), which is made possible by the lack of valves in this venous system, and by the proper use of gravity and positioning of the patient so that venous flow in the desired direction is accomplished. The rich connections between the cranial venous system and the vertebral venous system were beautifully depicted in 1828 by Breschet (reference 56), but this anatomic route remains largely unrecognized by the medical community till the present time.

Correct positioning of the patient so as to facilitate retrograde flow in the desired direction is utilized as part of the present invention to achieve improved delivery of radiolabeled etanercept and other large molecules to the brain, the eye, the retina, the auditory apparatus, the cranial nerves or the head from its injection point. Since the target is delivery of the large molecule to the brain, the eye, the retina, the auditory apparatus, the cranial nerves or the head, positioning following delivery utilizing head-down trendelenburg positioning, assists in delivering the large molecule to the target. In most cases, for delivery of a large molecule to the brain, the eye, the retina, the auditory apparatus, the cranial nerves or the head, interspinous injection is preformed overlying the posterior aspect of the cervical spine, in the interspinous region between the C4 and C8 spinal processes, followed by placement of the patient in the head-down trendelenburg position, usually in the prone position, if possible, since the large molecule is delivered, as described, to an area dorsal to the spine.

Batson's plexus may be used to introduce a variety of imaging molecules to the brain, retina, cranial nerves, and head via retrograde venous flow from Batson's plexus into the cranial venous sinuses and the intracranial venous system. This method bypasses the well known barrier which prevents large molecules introduced into the systemic circulation from reaching the brain (the BBB). The BBB prevents molecules larger than approximately 600 daltons from entering the brain via the systemic circulation. Virtually all biopharmaceuticals are larger than this. For example, etanercept has a molecular weight of 149,000 daltons, and insulin has a MW of 5,000 (compared with water which has a MW of 18). This method is particularly useful, therefore, for the administration of biologics, such as etanercept, erythropoietin, GM-CSF, ranibizumab, etc., whose size when delivered systemically prevents their efficient passage into the brain, retina, eye, and cranial nerves, but whose potency, because of their biologic origin, is extremely high. Effective delivery of these molecules to the brain, the retina, the eye, and the cranial nerves using the methods of the present invention thereby enables the treatment of a wide range of previously intractable disorders of the brain, the retina, and the nervous system, including those which are inflammatory; malignant; infectious; autoimmune; vascular; and degenerative.

The vertebral venous system is both anatomically and physiologically distinct from the venous system which drains the abdomen and thorax, which has been designated by others as the intracavitary venous system, with the vertebral venous system designated as the extracavitary venous system. Other nomenclature for the VVS also comes to mind, such as the valveless venous system, or the bi-directional venous system, but they are perhaps less suitable than the VVS. The VVS and the intracavitary venous system also share anastomoses, as has been discussed at length by Batson. Batson has also described the retrograde flow possible with the VVS, but has not proposed the possible use of the VVS as a route to deliver diagnostic compounds, nor has anyone else. Again, this retrograde route of delivery is uniquely possible utilizing the VVS because of the lack of venous valves.

Use of the vertebral venous system as a route to deliver radiolabeled etanercept to the retina, eye or optic nerve or spinal structures via retrograde venous flow is a novel new imaging method for imaging disorders of the brain, retina, eye or optic nerve.

This method allows the imaging of inflammatory or degenerative disorders of the retina and/or optic nerve, such as macular degeneration, diabetic retinopathy, glaucoma and retinitis pigmentosa, which involve excessive levels of TNF or which are mediated by VEGF. Excess TNF appears to have a direct deleterious effect on vision, and etanercept, delivered via the vertebral venous system, appears to have the ability to ameliorate this adverse effect. Perispinal administration of these biologics enables the biologic to reach the internal contents of the eye, including the choroidal vasculature and the retina, in diagnostic amounts, via retrograde flow within the cranio-vertebral venous system.

The methods of the present invention include the perispinal administration of the biologics of consideration herein (listed below), which can be accomplished in various ways, including transcutaneous interspinous injection, or catheter delivery into the epidural or interspinous space, which results in the biologics being delivered into the vertebral venous system and thence into the brain, retina, cranial nerves, and auditory apparatus in an improved amount which improves or enables functional imaging, depicting areas of inflammation or disease involvement, or of disease progression.

As defined herein, the auditory apparatus includes the cochlea, the auditory division of the eighth cranial nerve, and the central auditory pathways. Sensorineural hearing loss is one particular category of hearing loss and is caused by lesions of the cochlea and/or the auditory division of the eighth cranial nerve. Prior to this invention, treatment of this condition was primarily limited to the use of hearing aids.

Midline interspinous administration of etanercept has been demonstrated (see below) to produce improvement in hearing to individuals with certain forms of non-conductive hearing loss. In addition to percutaneous injection into the interspinous space, etanercept may also be delivered to the interspinous or epidural space by implantable catheter, with the catheter reservoir placed remotely, such as in the abdominal area.

The inventor first described improvement in hearing in a 73 y.o. patient after perispinal administration of etanercept for the treatment of sciatica in U.S. Pat. No. 6,423,321. The anatomic route which enables the efficient delivery of perispinal etanercept to the brain is identified by the inventor, and physical maneuvers to facilitate this process are described herein. For the purposes of this patent perispinal etanercept is distinguished from the use of etanercept delivered by subcutaneous administration at anatomic sites, such as the abdomen, thighs, and arms, which are remote from the spine.

Bevacizumab (Avastin™, Genentech) is a recombinant humanized monoclonal IgGI antibody that binds to and inhibits the biologic activity of human vascular endothelial growth factor (VEGF) and which may be useful for the treatment and imaging of retinal disorders which involve neovascularization. Bevacizumab has a molecular weight of 149,000 daltons and is therefore too large to readily cross the blood-brain barrier if administered systemically. Administration of bevacizumab via the vertebral venous system bypasses the blood-brain barrier and allows a therapeutic dose of bevacizumab to reach the retina, therefore enabling the treatment and imaging (if bevacizumab is radiolabeled or otherwise tagged) of retinal disorders which involve neovascularization, including macular degeneration and diabetic retinopathy. For this purpose bevacizumab may be administered via perispinal administration, thereby providing access of this monoclonal antibody to the VVS and therefore to the retina.

Pegaptanib and ranibizumab are two biologics which are antagonists of human vascular endothelial growth factor (VEGF) and which may be useful for the treatment of retinal disorders which involve neovascularization. Pegaptanib is a VEGF-neutralizing oligonucleotide aptamer which binds and sequesters VEGF, thereby preventing VEGF receptor activation. Ranibizumab is a recombinant humanized monoclonal antibody fragment with specificity for VEGF. Both pegaptanib and ranibizumab are too large to readily cross the blood-brain barrier or the blood-ocular barrier if administered systemically. They have both shown some efficacy in treating ocular neovascularization when administered by injection into the eye by the intravitreal route. Administration of these agents via the vertebral venous system bypasses the blood-brain barrier and the blood-ocular barrier and allows a diagnostic dose to reach the retina, therefore enabling the treatment of retinal disorders which involve neovascularization, including macular degeneration and diabetic retinopathy, without the necessity for intravitreal injection. For this purpose pegaptanib and ranibizumab may be administered via perispinal administration, thereby providing access of biologics to the VVS and therefore to the retina, the choroidal vessels, and the eye without requiring intravitreal injection. Additionally perispinal injection of these two biologics will enable effective delivery of these agents to the brain, thereby allowing the use of these agents for brain tumors and other clinical disorders which will respond positively to modulation of VEGF.

Perispinal administration for delivery of neuroactive molecules other than etanercept, including biologics, cytokines, anti-cytokines, hormones or drugs via the vertebral venous system, in a manner similar to that outlined herein, may be performed. The neuroactive compounds include the individual interleukins IL-1, IL-2, IL-4, IL-6, IL-10, or IL-13; interleukin 1 antagonists, such as IL-1 RA (Kineret®, Amgen) and IL-1 Trap; fusion proteins, such as IL-10 fusion protein or etanercept (Enbrel®, Immunex); other TNF antagonists, including certolizumab pegol, soluble TNF receptor type I or pegylated soluble TNF receptor type 1; human growth hormone and related biologics (recombinant human growth hormone, Humatrope® (somatropin) Eli Lilly & Co., Nutropin®/Nutropin AQ® (somatropin), Geref® (sermorelin) Serono, and Protropin® (somatrem) Genentech)); BDNF; erythropoietin (Epogen® (epoetin alpha) Amgen, Procrit® (epoetin alpha) Johnson & Johnson); G-CSF (Neupogen® (filgrastim), Amgen); GM-CSF; Intron® A (interferon alfa-2b) Schering-Plough; Avonex® (interferon beta-1a) Biogen; Alefacept (LFA-3/lgG1 human fusion protein, Amevive® Biogen); Epidermal growth factor; anti-EGF (ABX-EGF, Abgenix); transforming growth factor-beta (TGF-beta); NGF; bevacizumab (Avastin™, Genentech); Copaxone® (glatiramer acetate), pegaptanib or ranibizumab as discussed above; or other compounds with CNS, immune, or vascular activity.

In particular this invention involves the perispinal administration of radiolabeled monoclonal antibodies, such as trastuzumab, anti-tau antibodies, and antibodies directed against tumor antigens, and other biologics, including, but not limited to, radiolabeled etanercept and golimumab, and amyloid imaging agents, such as FDDNP, PIB, and radiolabeled anti-amyloid antibodies, such as AAB-001. Golimumab is currently in clinical development by Centocor/Schering-Plough for treatment of rheumatoid arthritis, with potential applications for uveitis, asthma, and Crohn's Disease. It may be described as a immunoglobulin G1, anti-(human tumor necrosis factor α) (human monoclonal CNTO 148 γ1-chain), disulfide with human monoclonal CNTO 148 κ-chain), dimer, and has CAS Registry number 476181-74-5. It is a fully human anti-TNF monoclonal antibody.

This invention involves the use of the above molecules delivered via the vertebral venous system for diagnostic imaging and/or therapeutic purposes. For example, use of a therapeutic dose of radiolabeled etanercept to a patient with intervertebral disc-related pain may result in a therapeutic clinical response. In addition imaging of the patient is facilitated by the radiolabeling, which may then reveal anatomic areas of excess TNF-alpha binding or production.

A biologic delivered via the vertebral venous system to the retina and the eye after perispinal administration is specifically included as an invention of the current patent.

The methods of the present invention are also distinguished from direct intrathecal administration of large molecules.

The large molecules of the current invention, when tagged for imaging by radiolabeling, include, but are not limited to, the following:
a. Colony-stimulating factors (including G-CSF, such as filgrastim, pegfilgrastim, and lenograstim; GM-CSF, including, but not limited to sargramostim and molgramostim; Erythroid growth factors, including, but not limited to: recombinant erythropoietin (EPO): epoetin alpha, darbepoetin alpha; and others.
b. TNF antagonists with a molecular weight greater than or equal to 2,000 daltons, including, but not limited to: golimumab, etanercept, infliximab, certolizumab (CDP 870, Cimzia®), CDP 571, onercept, pegylated soluble TNF receptor type I, soluble TNF receptor type I.
c. Interferons, interferon antagonists, and interferon fusion proteins, including, but not limited to: IL-1 Trap; Interferon alfa-2a, rDNA [Interferon alfa-2a—Roferon A; Interferon, alpha-2a, recombinant]; Interferon alfa-2a, rDNA, PEG-[Peginterferon alfa-2a—Pegasys; interferon alpha-2a, recombinant, pegylated]; Interferon alfa-2b, rDNA [Interferon alfa-2—Intron A; Interferon, alpha-2b, recombinant]; Interferon alfa-2b, rDNA, PEG-[Peginterferon alfa-2b—PEG-Intron Powder; interferon alpha-2b, recombinant, pegylated]; Interferon alfa, rDNA/BioPartners [Interferon alpha, recombinant]; Interferon alfacon-1, rDNA [Interferon alfacon-1—Infergen; consensus interferon, recombinant]; Interferon beta-1a, rDNA/Biogen [Interferon beta-1—Avonex [recombinant]]; Interferon beta-1a, rDNA/Serono [Interferon beta-1a—Rebif [recombinant]]; Interferon betaser, rDNA/Berlex [Interferon beta-1b—Betaseron] (Betaseron has a MW of 18500 daltons); 2-166-Interferon beta1 (human fibroblast reduced), 17-L-serine-; interferon betaser, recombinant]; Interferon gamma, rDNA [Interferon gamma-1b—Actimmune; [recombinant]]; Interleukin-1ra, rDNA [Anakinra—Kineret; interleukin-1 receptor antagonist; IL-1i]; Interleukin-2, rDNA [Aldesleukin—Proleukin; des-alanyl-1, serine-125 interleukin-2, recombinant; IL-2]; Interleukin-2/diphtheria toxin, rDNA [Denileukin diftitox—ONTAK; Interleukin-2 Fusion Protein; DAB389IL-2; interleukin-2/diphtheria toxin fusion protein, recombinant]; MRA (Roche, Chugai), a humanized anti-IL-6 receptor monoclonal antibody; Interleukin-2 receptor Mab, rDNA/Novartis [Basiliximab—Simulect; Interleukin-2 alpha receptor monoclonal antibody, recombinant]; Interleukin-2 receptor Mab, rDNA/Roche [Daclizumab—Zenapax; Interleukin-2 alpha receptor monoclonal antibody, recombinant]; Interleukin-11, rDNA [Oprelvekin—Neumega; des-Pro Interleukin-11, recombinant; des-Pro IL-11]; IL-6; IL-12; anti-IL-6; and anti-IL-12. As a general rule, interferons have molecular weights ranging from 15,000 to 21,000 daltons.
d. Antibiotics with a molecular weight of 2,000 daltons or greater;
e. Cancer chemotherapeutic agents, with a molecular weight greater than or equal to 2,000, including those from the following classes:
   i. Monoclonal antibodies (mAb): including, but not limited to:
      1. Rituximab, a chimeric murine mAb against the CD20 antigen on B-lymphoma cells.
      2. Epratuzumab, a humanized mouse anti-CD22 mAb.
      3. Alemtuzumab, a humanized mAb against CD 52 on B and T lymphoma cells.
      4. Natalizumab, a humanized mAb against the alpha4 subunit of the alpha4 Beta1 and Beta 7 integrins.
      5. Trastuzumab
   ii. Conjugates: Monoclonal antibody-drug, -toxin, or -radionuclide conjugates. These antibodies recognize specific antigenic determinants on malignant cells and their conjugates provide selective toxicity to those cells. A monoclonal antibody conjugate, for the purpose of this invention, is defined as a monoclonal antibody which is conjugated to either a drug, a toxin (such as diptheria toxin) or a radionuclide. These conjugates are particularly suited to perispinal administration, since they are extremely effective, even at low concentration, due to their biologic origin, and can be effectively delivered to the brain or to a brain tumor or lymphoma via the VVS by retrograde venous delivery into the brain. Therefore this class of therapeutic is effective for treating malignant tumors of the brain, either primary, such as glioblastoma multiforme, or metastatic, and for treating CNS lymphomas. These agents include yttrium-90 ibritummomab tiuxetan (Zevalin®) and iodine-131 tositumomab (Bexxar®) which are both murine mAbs against CD20 antigen that are conjugated to a radioactive source and thus selectively deliver radiation to tumors expressing the CD20 antigen (primarily expressed on B-lymphomas).

The above methods detailed for large molecules may be used identically for molecules with a MW of less than 2,000 daltons. The rationale for doing this is that many of these molecules, despite their smaller size, still have difficulty traversing the blood-brain barrier if administered systemically; or perispinal delivery without direct intrathecal injection results in more efficient delivery of these smaller molecules to the brain, the eye, or the auditory apparatus than does systemic or oral delivery. Perispinal administration and delivery to the brain, the eye, or other structures of the head thereby has the advantage of more efficient delivery across the BBB. For example the taxanes, which include paclitaxel (Taxol®) and docetaxel (Taxotere®) have very low BBB penetration when given systemically, despite their respective MW of 854 and 862. Doxorubicin has poor BBB penetration when given systemically despite its MW of 544. Methotrexate and Amphotericin B have poor BBB penetration when given systemically, despite a MW of 454 and 924, respectively, and are often administered intrathecally for CNS use. The perispinal extrathecal methods of the present invention are distinguished from direct intrathecal injection.

Perispinal extrathecal administration of anti-cancer agents which are radiolabeled may serve a two-fold purpose. This method of administration facilitates or enables delivery of these molecules to sites across the blood-brain, blood-eye, and blood-nerve barrier i.e. the brain, the eye, the spinal cord, etc. If these radiolabeled anti-cancer agents are administered in microdoses this may facilitate imaging of cancer or cancer metastases while reducing or eliminating toxicity. If administered in larger, therapeutic doses there is both an enhanced therapeutic effect and facilitated imaging.

Perispinal extrathecal administration of radiolabeled small molecules may also be used as PET imaging agents to image the brain or spinal structures. These various drugs may be radiolabeled with either [11C] or [18F] to facilitate PET or microPET imaging, or with [123I] or [125I] to facilitate SPECT or microSPECT imaging. For use with these imaging methods microdoses of these agents can be used i.e. less than 1/100 of the smallest therapeutic dose normally used at a maximum dose of 100 micrograms, with a usual dose in the range of 0.5 to 100 micrograms for imaging purposes. With respect to the small molecules of the present invention, they may be categorized as follows:
1. Cancer chemotherapeutic agents, with a molecular weight less than 2,000, including, but not limited to those from the following classes: (Clinical use: treatment of tumors of the central nervous system or the orbit utilizing perispinal administration without direct intrathecal injection of the following):
   i. Alkaloids: vincristine, vinblastine, vindesine, paclitaxel (Taxol®), docetaxel, etoposide, teniposide.
   ii. Alkylating agents: nitrogen mustards, nitrosureas, cyclophosphamide, thiotepa, mitomycin C, dacarbazine.
   iii. Antibiotics: Actinomycin D, daunorubicin, doxorubicin, idarubicin, mitoxanthrone, bleomycin, mithramycin.
   iv. Antimetabolites: methotrexate, 6-mercaptopurine, pentostatin, 5-fluorouracil, cytosine arabinoside, fludarabine, 2-CDA.
   v. Platinum compounds: Cisplatin.
   vi. Others: tamoxifen (MW 563), flutamide (MW 276), anastrozole (MW 293), gefitinib (Iressa®) and erlotinib (Tarceva®) (MW 429).
2. Antibiotics: including, but not limited to cephalosporins, tetracyclines, macrolides, fluroquinolones.
3. Anti-parkinson drugs: (Clinical use: brain and spinal cord imaging): including, but not limited to levodopa, carbidopa, bromocriptine, selegiline, and dopamine.
4. Anti-psychotic agents: (Clinical use: brain and spinal cord imaging): haloperidol, Prolixin®, Moban®, Loxitane®, Serentil®, Trilafon®, Clozaril®, Geodon®, Risperdal®, Seroquel®, and Zyprexa®.
5. Antidepressants: (Clinical use: brain and spinal cord imaging), utilizing perispinal administration without direct intrathecal injection of the following): including, but not limited to tricyclics, tetracyclics, trazadone, and SSRIs.
6. Anticonvulsants: (Clinical use: brain and spinal cord imaging): including, but not limited to, Valium®, phenytoin, other hydantoins, barbiturates, gabapentin, lamotrigine, carbamazepine, topiramate, valproic acid, and zonisamide.
7. Opiates and opioids: (Clinical use: brain and spinal cord imaging), utilizing perispinal administration without direct intrathecal injection of the following): including, but not limited to morphine, oxycodone, other opiates and opioids, including oxycontin and methadone.

Perispinal extrathecal administration is distinguished from intrathecal administration because extrathecal administration is both safer (no dural puncture, therefore no risk of CSF leak; less risk of hemorrhage; no risk of spinal cord traumatic injury; less risk of hemorrhage and infection) and is more effective at delivering the imaging molecule into the VVS. The dural barrier, once crossed, will contain the imaging molecule within the CSF. CSF flow from the spinal cord to the brain is slow. In contrast retrograde flow to the brain via the VVS is much more rapid.

For the purposes of this discussion, “perispinal” means in the anatomic vicinity of the spine, but outside of the intrathecal space. For this discussion “anatomic vicinity” is generally defined as within 10 centimeters, or functionally defined as in close enough anatomic proximity to allow the diagnostic molecules of consideration herein to reach diagnostic concentration when administered directly to this area without necessitating direct intrathecal delivery.

Perispinal administration for delivery of large molecules, including biologics, cytokines, anti-cytokines, hormones or drugs via the vertebral venous system, in a manner as outlined herein, may be performed. The compounds could include interleukins, cytokines, interferons, drugs, growth factors, VEGF inhibitors, monoclonal antibodies, fusion proteins, anti-angiogenic agents, chemotherapeutic agents, cytostatic agents, cancer therapeutics, or other agents useful for imaging for which delivery by perispinal administration without direct intrathecal injection would be beneficial.

One of the advantages of perispinal delivery into the interspinous space is that administration is simplified. This route is simple and safe. Hemorrhage due to the use of long or large bore needles is minimized because perispinal administration, by the subcutaneous route, requires only a short, narrow bore needle. Time-consuming and difficult epidural injection is not necessary. Local perispinal administration also has the advantage of providing a depot of medication in the surrounding tissue, which will provide levels of medication to the imaging site for a prolonged period of time. This decreases the necessity for another injection of medication. Additionally, administering medication locally limits the exposure of the medication to the systemic circulation, thereby decreasing renal and hepatic elimination of the medication, and decreasing exposure of the medication to systemic metabolism. All of these factors tend to increase the imaging half-life of the administered large molecule. Taken together, all of these forms of perispinal administration have significant clinical advantages over the various forms of systemic administration customarily used to deliver large molecules systemically. For example, intravenous administration (as conventionally performed, by infusion into the caval venous system) of infliximab is a systemic route of administration, as defined herein, and is distinguished from perispinal administration as a method to reach the brain (predominantly via the VVS) as defined herein.

For the sake of this invention, the following definitions also apply: perilesional is defined as in anatomic proximity to the site of the pathologic process being treated; and peridural is defined as in anatomic proximity to the dura of the spinal cord, but specifically excluding intrathecal injection. The “interspinous route” for the purposes of this patent, is defined as parenteral injection through the skin in or near the midline, in the interspace between two spinous processes.

This invention is distinguished from the prior art in a variety of ways, including the use and description of novel and useful new uses, methods of use, and concepts involving large molecules, including:
1. Novel uses of perispinal administration to enhance delivery of a large molecule to the brain, the eye, the retina, the auditory apparatus, the cranial nerves or the head; and
2. Novel methods of use of large molecules; and
3. Novel concepts, including:
   a. Perispinal (extrathecal) administration distinguished from systemic forms of administration and intrathecal administration;
   b. The use of the vertebral venous system to deliver large molecules to the bone brain, the eye, the retina, the auditory apparatus, the cranial nerves or the head;
   c. The use of physical maneuvers to facilitate delivery of imaging molecules to the brain, the eye, the retina, the auditory apparatus, the cranial nerves or the head;
   d. The use of physical positioning to influence the direction of venous flow within the vertebral venous system and thereby deliver imaging molecules to the spine, the spinal nerve roots, the intervertebral discs, the spinal cord, brain, the eye, the retina, the auditory apparatus, the cranial nerves or the head;
   e. The use of retrograde venous perfusion to deliver imaging molecules to the brain, the eye, the retina, the auditory apparatus, the cranial nerves or the head;
   f. The use of retrograde venous perfusion via the vertebral venous system to facilitate delivery of molecules to the brain, the spine, the spinal cord, the spinal nerve roots, the dorsal root ganglia, the intervertebral discs, the eye, the retina, the auditory apparatus, the cranial nerves or the head;
   g. The use of the vertebral venous system as a “back door” to facilitate delivery of imaging molecules to the brain, the eye, the retina, the auditory apparatus, the cranial nerves or the head;
   h. The use of perispinal administration to introduce a large molecule into the vertebral venous system;
   i. The use of perispinal administration to efficiently deliver large molecules to the brain, the eye, the retina, the auditory apparatus, the cranial nerves or the head.

The same methods described for the named large molecules (such as pegfilgrastim) of this invention also apply to other large molecules with a molecular weight of 2,000 daltons or greater, which may be given by perispinal administration.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

(Experimental results compiled by the inventor illustrating the efficacy of perispinal administration of a biologic are described below. More specifically, these results illustrate the ability of interspinous injection to lead to delivery of a biologic to the VVS, and thereafter to the brain, utilizing the methods of the present invention).

### EXPERIMENTAL RESULTS

An IRB-approved clinical trial utilizing perispinal etanercept for treatment of Alzheimer's Disease was begun by the inventor in 2004 and clinical data is available on the first 15 consecutive patients who completed more than three weeks of the clinical trial, through Nov. 7, 2005, although the clinical trial is ongoing. Data on the 6 month results is now available. A summary of the study follows:

### Patients

Patients residing in the community, who had previously been diagnosed with Alzheimer's Disease by a board-certified neurologist and were clinically declining despite treatment, were recruited, without age restriction, for inclusion into a six month open-label clinical trial utilizing perispinally administered etanercept. Inclusion required that the patient meet the NINCDS-ADRDA Criteria for probable Alzheimer's disease[1]; be accompanied by a reliable caregiver; and have a previously performed MRI or CT scan consistent with a primary diagnosis of AD. All recruited patients also met the DSM-IV criteria for AD[2]. Patients were excluded if they had any of the following: active infection, multiple sclerosis (or any other demyelinating disorder), pregnancy, uncontrolled diabetes mellitus, tuberculosis, history of lymphoma, or congestive heart failure. In addition, female subjects who were premenopausal, fertile, or not on acceptable birth control; and patients with a white blood cell count <2500, hematocrit <30, or a platelet count <100,000 were excluded. Patients with vascular dementia, clinically significant neurologic disease other than Alzheimer's, or a score greater than 4 on the modified Hachinski Ischemic Rating Scale[3] were excluded. Additionally, to be eligible for study inclusion, the dosage of all CNS-active medications was required to be unchanged in the four weeks prior to study initiation and during the entire course of the clinical trial.

### Study Design

Patients received etanercept (Immunex Corp.) as a solution in sterile water given by midline perispinal interspinous injection in the posterior cervical area (as previously described [4]) utilizing a thin (27 gauge) needle, followed by head down Trendelenburg positioning, once or twice per week, at a total dose ranging from 25 mg to 50 mg per week (0.5-2 cc of solution) on an open-label basis. The initial dose used was 25 mg once per week, which was modified as needed. The trial was approved by a central institutional review board. The eligible patients and their responsible caregivers provided written informed consent.

### Efficacy Variables

The primary efficacy variables for cognition were three measures: the Alzheimer's Disease Assessment Scale-cognitive subscale (ADAS-Cog); the Severe Impairment Battery; the Mini-Mental State Examination (MMSE).

Patients were assessed at baseline (treatment day zero) and monthly thereafter. All patients were assessed with the MMSE. Patients with mild and moderate AD were assessed with ADAS-Cog. Patients with severe dementia were assessed with the SIB.

Measures of safety included measurement of vital signs and recording of adverse events.

### Results

### Study Population and Dosage

All data from all 15 patients who completed at least one follow-up evaluation time-point were analyzed. All of these patients completed the first six months of treatment. Treatment response data were unavailable for two patients, in addition to the above 15, who dropped out for non-medical reasons prior to their first monthly evaluation; these two patients were excluded from analysis. One patient whose dementia was borderline between moderate and severe was assessed with both ADAS-Cog and SIB, in addition to MMSE. The baseline characteristics of the 15 patient study population are presented in Table 1. The average dosage for the study cohort was 32±12 mg per week (n=15), and the average frequency of dosing was 1.07 times per week.

### Statistical Analysis

The main efficacy analysis at 6 months is based on all 15 patients who have baseline and follow-up data.

The MMSE, ADAS-Cog, and the SIB are considered as the primary outcome measures at the end of the three month follow-up assessment. Mixed Model Linear Regression (MMLR) analyses were used to assess improvement in disease over time, as evaluated by the four outcome measures. In each analysis, time (baseline, 1, 2, 3, 4, 5 and 6 months) was entered as a fixed variable. The models were also specified with random intercepts, as the participants in this study varied across the spectrum of severity at baseline because recruitment was not limited to a range of severity. Missing data points are treated as missing and are not estimated; this was an observed data analysis.

Data were analyzed using statistical analysis software SPSS (Version 11.0.3 for Mac OS X, SPSS Inc., Chicago, Ill., USA), with p<0.05 indicative of statistical significance.

### Efficacy

The results of treatment through six months and the statistical analysis are presented in Table 1.

TABLE 1
Summary of Mixed Model Linear Regression (MMLR) results following initiation of
perispinal etanercept.

| | | Mean | Mean | Mean | Mean | Mean | Mean | |
|---|---|---|---|---|---|---|---|---|
| | | change | change | change | change | change | change | |
| | Baseline | at 1 | at 2 | at 3 | at 4 | at 5 | at 6 | |
| | Mean | month | months | months | months | months | months | Regression Analyses |
| Measure (n) | (SD) | (SD) | (SD) | (SD) | (SD) | (SD) | (SD) | Results |
| MMSE (15) | 18.2 | −.29 | +1.07 | +1.87 | +2.00 | +1.93 | +2.13 | F (1.84) = 39.00, |
| | (8.8) | (1.82) | (2.01) | (1.99) | (2.13) | (2.34) | (2.23) | p < .001 |
| ADAS-cog (11) | 20.85 | −4.28 | −4.64 | −4.67 | −7.14* | −4.52 | −5.48 | F (1.61) = 11.72, |
| | (10.5) | (3.44) | (4.36) | (5.97) | (4.51) | (4.80) | (5.08) | p < .002 |
| SIB (5) | 62.5 | +4.67 | +8.2 | +11.75 | +13.6 | +13.0 | +16.6 | F (1.26) = 22.60, |
| | (28.05) | (6.35) | (3.56) | (6.45) | (10.89) | (13.69) | (14.52) | p < .001 |

Caption: Baseline raw group mean and standard deviations are presented with the mean change and SD (each participant compared to their respective baseline performance) for the 6 subsequent follow-up months.
Note:
For the ADAS-Cog, lower scores indicate clinical improvement.
*Note 2: reduced n = 7 at this time point.
SD = Standard Deviation

TABLE 2
Patient Characteristics, at baseline, prior to
perispinal etanercept treatment.

| Characteristic | Mean ± SD | Range |
|---|---|---|
| Age, in yrs. | 76.7 ± 10.9 | 52, 94 |
| Female, % (n) | 60% (9) | — |
| Duration of symptoms, in mos. | 43.1 ± 37.9 | 8, 120 |
| ADAS-Cog score (n = 11) | 20.8 ± 10.5 | 7.3, 41 |
| SIB score (n = 5) | 62.5 ± 28.05 | 28, 92 |
| MMSE score (n = 15) | 18.2 ± 8.8 | 0, 29 |
| Prior Treatments: | | |
| Memantine, % (n) | 73% (11) | — |
| Duration prior to Etanercept, | 10.6 ± 4.0 | 1.5, 15 |
| in mos. | | |
| Donepezil, % (n) | 47% (7) | — |
| Duration prior to Etanercept, | 44.7 ± 47.9 | 10, 120 |
| in mos. | | |
| Rivastigmine, % (n) | 27% (4) | — |
| Duration prior to Etanercept, | 5.6 ± 3.3 | 1, 8 |
| in mos. | | |
| Galantamine, % (n) | 13% (2) | — |
| Duration prior to Etanercept, | 40.5 ± 6.4 | 36, 45 |
| in mos. | | |
| Only 1 of the above, % (n) | 40% (6) | — |
| Memantine + a cholineserase | 60% (9) | — |
| inhibitor, % (n) | | |

### (End of Experimental Results).

### Preferred Embodiments

In one preferred embodiment PET imaging of the brain is performed in a human with dementia following a perispinal injection of [11C]PIB, delivered by midline transcutaneous injection overlying the spine in the lower posterior neck area, with the patient sitting and head flexed forward, with immediate placement of the patient in the prone position with the plane of the examination table directed head downward about 15 degrees after the injection, and maintenance of the patient in this modified Trendelenburg prone position for several minutes after injection.

In another preferred embodiment PET imaging of the brain is performed in a human with a history of cancer following a perispinal injection of radiolabeled trastuzumab, delivered by midline transcutaneous injection overlying the spine in the lower posterior neck area, with the patient sitting and head flexed forward, with immediate placement of the patient in the prone position with the plane of the examination table directed head downward about 15 degrees after the injection, and maintenance of the patient in this modified Trendelenburg prone position for several minutes after injection.

In another preferred embodiment PET imaging of the brain is performed in a human with dementia following a perispinal injection of [18F]FDDNP, delivered by midline transcutaneous injection overlying the spine in the lower posterior neck area, with the patient sitting and head flexed forward, with immediate placement of the patient in the prone position with the plane of the examination table directed head downward about 15 degrees after the injection, and maintenance of the patient in this modified Trendelenburg prone position for several minutes after injection.

In another preferred embodiment PET imaging of the brain is performed in a human with dementia following a perispinal injection of [11C]PK11195, delivered by midline transcutaneous injection overlying the spine in the lower posterior neck area, with the patient sitting and head flexed forward, with immediate placement of the patient in the prone position with the plane of the examination table directed head downward about 15 degrees after the injection, and maintenance of the patient in this modified Trendelenburg prone position for several minutes after injection.

In another preferred embodiment PET imaging of the brain is performed in a human with dementia following a perispinal injection of [11C]DAA1106, delivered by midline transcutaneous injection overlying the spine in the lower posterior neck area, with the patient sitting and head flexed forward, with immediate placement of the patient in the prone position with the plane of the examination table directed head downward about 15 degrees after the injection, and maintenance of the patient in this modified Trendelenburg prone position for several minutes after injection.

In another preferred embodiment PET imaging of the spine is performed in a human with back pain following a perispinal injection of [11C]-labeled etanercept, delivered by midline transcutaneous injection overlying the spine in the region of the L4-5 interspace, with the patient in the left lateral decubitus position, with immediate placement of the patient in the prone position with the plane of the examination table horizontal after the injection, and maintenance of the patient in this flat prone position for several minutes after injection.

In another preferred embodiment SPECT imaging of the spine is performed in a human with back pain following a perispinal injection of [125I]-labeled etanercept, delivered by midline transcutaneous injection overlying the spine in the region of the L4-5 interspace, with the patient in the left lateral decubitus position, with immediate placement of the patient in the prone position with the plane of the examination table horizontal after the injection, and maintenance of the patient in this flat prone position for several minutes after injection.

In another preferred embodiment PET imaging of the brain is performed in a human with dementia following a perispinal injection of a microdose of radiolabeled AAB-001, delivered by midline transcutaneous injection overlying the spine in the lower posterior neck area, with the patient sitting and head flexed forward, with immediate placement of the patient in the prone position with the plane of the examination table directed head downward about 15 degrees after the injection, and maintenance of the patient in this modified Trendelenburg prone position for several minutes after injection.

In another preferred embodiment PET imaging of the brain is performed in a human with dementia following a perispinal injection of a microdose of radiolabeled AAB-002, delivered by midline transcutaneous injection overlying the spine in the lower posterior neck area, with the patient sitting and head flexed forward, with immediate placement of the patient in the prone position with the plane of the examination table directed head downward about 15 degrees after the injection, and maintenance of the patient in this modified Trendelenburg prone position for several minutes after injection.

In another preferred embodiment interspinous injection is accomplished by injection through the skin.

### Tagging of Therapeutic Agents to Facilitate Diagnostic Imaging

The methods of the present invention involve the use of therapeutic molecules to facilitate the use of these agents for imaging purposes. These agents are delivered by perispinal administration. Their use as diagnostic agents is facilitated by alteration of these molecules, generally by the addition of radioactive tracers or other methods of “tagging” of these therapeutic molecules to facilitate their use as imaging agents. This tagging will often involve standard methods of radiolabeling. Standard radiolabeling methods used include, but are not limited to, radioactive iodination, use of technetium-99, use of [123-I]-labeled ligands, and radiolabeling with either [11C] or [18F]. In addition, tagging may involve the conjugation of paramagnetic particles with large molecules to enhance MRI imaging, or the use of fluorescein-labeling to facilitate optical imaging, particularly useful in retinal imaging and retinal diseases.

Amyloid imaging of the brain using PET is facilitated by using microdoses of [18F]FDDNP, [11C]PIB or microdoses of radiolabeled anti-amyloid antibodies, such as [11C] or [18F]-labeled AAB-001 (a humanized anti-amyloid monoclonal antibody being developed by Elan/Wyeth) or [11C] or [18F]-labeled AAB-002 (another anti-amyloid monoclonal antibody being developed by Elan/Wyeth). For the purposes of this invention the doses of the anti-amyloid antibodies used for imaging are microdoses i.e. less than 1/100 of the dose used by conventional intravenous administration for therapeutic use. In the case of radiolabeled AAB-001 and radiolabeled AAB-002 the dose used for brain imaging of amyloid using perispinal extrathecal administration is between 1/1000 and 1/100 of the normal therapeutic dose. PET imaging of activated microglia in diseased brains or the spinal cord is facilitated by using [11C]PK11195 or [11C]DAA1106, which label the peripheral benzodiazepine-binding sites that are selectively expressed on activated microglia.

SPECT imaging of Parkinson's disease is performed using [123-I]-labeled beta-CIT. In addition, Parkinson's disease can be imaged using radiolabeled monoclonal antibodies or antibody fragments against alpha-synuclein e.g. PET imaging of Parkinson's using an [11C]-radiolabeled anti-alpha-synuclein humanized monoclonal antibody.

Various [11C] labeled ligands have been developed for PET imaging, most of which involve dopamine or serotonin receptors or transporters. These PET ligands may be administered in microdose amounts by perispinal extrathecal delivery to improve their use in brain imaging, and are useful in imaging the brain in schizophrenia.

The methods of the present invention, including specifically perispinal extrathecal administration of large molecules radiolabeled with [11C] or [18F], followed by Trendelenburg positioning, enable the successful expansion of the number of radioligands suitable for PET imaging of both the brain and the spinal cord.

Radiolabeling and use of PET imaging of the brain enables the use of microdosing i.e. dosing at less than one-hundredth of the pharmacologically active dose and not more than 100 ug. The use of microdosing facilitates the approval of human microdosing trials and is used to facilitate the development of the novel methods of the present invention. Microdosing and PET imaging of the brain can be used even for molecules of the present invention, such as etanercept, which are currently FDA-approved for therapeutic use. For the present invention etanercept may be radiolabeled with 99-technetium, [11C], [18F], [123I], [125I], or other suitable radioligand and administered in usual therapeutic quantities (e.g. 10 mg) or microdosed for PET imaging in a range near 100 ug or less. [11C] and [18F] are positron emitters which faciliate PET imaging; [123I] and [125I] are single photon emitters which facilitate SPECT imaging.

### Imaging Categories

The following is a short listing of the main categories for which imaging with radiolabeled large molecules delivered by the perispinal route without direct intrathecal injection will be useful:

### Brain Imaging.

The methods of the present invention involve the use of therapeutic molecules to facilitate the use of these agents for imaging purposes. These agents are delivered by perispinal administration. Their use as diagnostic agents is facilitated by alteration of these molecules, generally by the addition of radioactive tracers or other methods of “tagging” of these therapeutic molecules to facilitate their use as imaging agents. This tagging will often involve standard methods of radiolabeling. Standard radiolabeling methods used include, but are not limited to, radioactive iodination, use of technetium-99, use of [123-I]-labeled ligands, and radiolabeling with either [11C] or [18F]. In addition, tagging may involve the conjugation of paramagnetic particles with large molecules to enhance MRI imaging, or the use of fluorescein-labeling to facilitate optical imaging, particularly useful in retinal imaging and retinal diseases.

Amyloid imaging of the brain using PET is facilitated by using microdoses of [18F]FDDNP, [11C]PIB or microdoses of radiolabeled anti-amyloid antibodies, such as [11C] or [18F]-labeled AAB-001 (a humanized anti-amyloid monoclonal antibody being developed by Elan/Wyeth) or [11C] or [18F]-labeled AAB-002 (another anti-amyloid monoclonal antibody being developed by Elan/Wyeth). For the purposes of this invention the doses of the anti-amyloid antibodies used for imaging are microdoses i.e. less than 1/100 of the dose used by conventional intravenous administration for therapeutic use. In the case of radiolabeled AAB-001 and radiolabeled AAB-002, the dose used for brain imaging of amyloid using perispinal extrathecal administration is between 1/1000 and 1/100 of the normal therapeutic dose. PET imaging of activated microglia in diseased brains or the spinal cord is facilitated by using [11C]PK11195 or [11C]DAA1106, which label the peripheral benzodiazepine-binding sites that are selectively expressed on activated microglia.

SPECT imaging of Parkinson's disease is performed using [123-I]-labeled beta-CIT. In addition, Parkinson's disease can be imaged using radiolabeled monoclonal antibodies or antibody fragments against alpha-synuclein e.g. PET imaging of Parkinson's using an [11C]-radiolabeled anti-alpha-synuclein humanized monoclonal antibody.

Various [11C] labeled ligands have been developed for PET imaging, most of which involve dopamine or serotonin receptors or transporters. These PET ligands may be administered in microdose amounts by perispinal extrathecal delivery to improve their use in brain imaging, and are useful in imaging the brain in schizophrenia.

The methods of the present invention, including specifically perispinal extrathecal administration of large molecules radiolabeled with [11C] or [18F], followed by Trendelenburg positioning, enable the successful expansion of the number of radioligands suitable for PET imaging of both the brain.

Radiolabeling and use of PET imaging of the brain enables the use of microdosing i.e. dosing at less than one-hundredth of the pharmacologically active dose and not more than 100 ug. The use of microdosing facilitates the approval of human microdosing trials and is used to facilitate the development of the novel methods of the present invention. Microdosing and PET imaging of the brain can be used even for molecules of the present invention, such as etanercept, which are currently FDA-approved for therapeutic use. For the present invention etanercept may be radiolabeled with 99-technetium, [11C], [18F], [123I], [125I], or other suitable radioligand and administered in usual therapeutic quantities (e.g. 10 mg) or microdosed for PET imaging in a range near 100 ug or less. [11C] and [18F] are positron emitters which faciliate PET imaging; [123I] and [125I] are single photon emitters which facilitate SPECT imaging.

Perispinal administration without direct intrathecal injection enables large molecules to effectively cross the blood-brain barrier and reach the brain. If radiolabeled then brain imaging is facilitated. Radiolabeling with positron-emitters facilitates PET imaging; radiolabeling with single photon emitters facilitates SPECT imaging; radiolabeling with other agents will facilitate gamma-camera imaging. Use of various large molecules will facilitate functional and/or molecular imaging. For example, use of radiolabeled anti-amyloid antibodies will reveal the distribution of amyloid deposits in the brain; use of radiolabeled anti-tau antibodies will reveal the distribution of tau in the brain; use of radiolabeled antibodies directed against tumor antigens can reveal the distribution of tumors, including primary and metastatic brain cancers and lymphomas in the brain. Visualization of the distribution and extent of these structures can reveal the extent of disease or disorder, and is useful in gauging the response to treatment. In addition, these methods, if used for anti-cancer agents, or anti-lymphoma agents, including, but not limited to, rituximab, trastuzumab and anti-angiogenesis agents, such as bevacizumab, will allow achievement of therapeutic concentrations of these agents in the brain, thereby facilitating or enabling treatment, in addition to the diagnostic usefulness of these methods.

The methods detailed herein are particularly useful for neurodegenerative diseases, including, but not limited to, Alzheimer's Disease, other forms of dementia, Parkinson's Disease, Huntington's Disease, amyotrophic lateral sclerosis, and multiple sclerosis. The radiolabeled large molecules suitable for perispinal extrathecal administration to enhance brain delivery include, but are not limited to, etanercept, golimumab, certolizumab pegol and other anti-TNF molecules (as illustrated by the experimental results for etanercept included herein), MRA (Roche, Chugai), a humanized anti-IL-6 receptor monoclonal antibody; anti-IL-1 molecules; immune globulin (such as IVIG, Baxter, being a mixture of immune globulins, including anti-amyloid antibodies), AAB-001, AAB-002, other anti-amyloid antibodies, anti-tau antibodies, interferons, and other large molecules with immune activity.

Spine and spinal cord imaging. Perispinal administration without direct intrathecal injection enables large molecules to effectively cross the blood-dural barrier and reach the spinal cord. If the large molecule is radiolabeled then imaging of the spinal cord and/or related spinal structures is facilitated. Radiolabeling with positron-emitters facilitates PET imaging; radiolabeling with single photon emitters facilitates SPECT imaging; radiolabeling with other agents will facilitate gamma-camera imaging. As with brain imaging, these methods are useful for spine and spinal cord tumors, including cancer and lymphoma.

Eye imaging. Perispinal administration without direct intrathecal injection enables large molecules to effectively cross the blood-eye barrier and reach the retina. If the large molecule is radiolabeled then imaging of the retina is facilitated. Radiolabeling with positron-emitters facilitates PET imaging; radiolabeling with single photon emitters facilitates SPECT imaging; radiolabeling with other agents will facilitate gamma-camera imaging. Functional imaging with large molecules, including VEGF antagonists (including bevacizumab, pegaptanib, or ranibizumab), and TNF antagonists (etanercept, infliximab, certulizumab pegol, and adalimumab), which have been fluorescein-labeled, is useful for imaging various retinal diseases, including retinal neovascularization, macular degeneration, diabetic retinopathy, and retinitis pigmentosa. Alternatively these large molecules, including VEGF antagonists (including bevacizumab, pegaptanib, or ranibizumab), and TNF antagonists (etanercept, infliximab, certolizumab pegol, and adalimumab), may be radiolabeled and the retina imaged with microPET, or microSPECT scanners.

Malignant Tumors metastatic to the spine: Malignant tumors metastatic to the spine may be imaged by the use of biologics delivered via the VVS. Access to the VVS may be accomplished by perispinal administration, in the general manner as described herein for etanercept. The inventor has found that perispinal etanercept is effective for the treatment of selected patients with cancer metastases to the spine (see reference 67). This invention includes any of the following molecules used individually: etanercept, golimumab, certolizumab pegol or pegsunercept; and, additionally, includes other biologic TNF antagonists, including infliximab, when delivered by perispinal extrathecal administration. Diagnostic imaging is facilitated if these anti-TNF agents are radiolabeled, and, if PET imaging is utilized, microdoses of these large molecules are effective to enable imaging.

Malignant intracranial tumors. This category includes both primary brain tumors, such as glioblastoma multiforme and tumors metastatic to the brain, all of which involve excess VEGF and/or the participation of VEGF-mediated angiogenesis, or immune mechanisms in their pathogenesis. Treatment of patients with these disorders with perispinal administration without direct intrathecal injection of a large molecule which inhibits VEGF; or which is directly toxic to a tumor, including, but not limited to monoclonal antibodies, or monoclonal antibody-antitumor conjugates; or which otherwise positively affects immune mechanisms; including, but not limited to such large molecules as etanercept, certolizumab pegol, IL-1 Trap, Kineret®, bevacizumab, pegaptanib, ranibizumab, Zevalin®, Mylotarg®, Campath®, HumaSpect®, panitumumab, trastuzumab, Ontak®, Simulect®, Zenapax®, leads to reduced tumor growth, tumor death, and/or slowing of disease progression. CNS lymphomas and other CNS malignancies may be treated by perispinal administration without direct intrathecal injection of rituximab, temozolomide, yttrium-90 ibritummomab tiuxetan, iodine-131 tositumomab, epratuzumab, alemtuzumab, or natalizumab. Radiolabeling of those large molecules mentioned above allows their use for diagnostic purposes. While conventional doses are used for therapeutic purposes, microdoses (less than 1/100 of the lowest usual therapeutic dose, and less than 100 micrograms) are used for PET imaging, which is facilitated if radiolabeling is performed with a positron emitter, such as [11C] or [18F]. Avoidance of intrathecal use is safer, has fewer side effects, avoids CSF leak from a dural tear, and eliminates the need for intrathecal delivery systems, such as pumps. Small molecules may also be administered by perispinal delivery without direct intrathecal injection as discussed in a preceding section. Perispinal delivery of small molecules allows the achievement of a higher concentration of the small molecule in the brain and therefore in an intracranial malignant tumor. This is particularly advantageous for small molecules which have therapeutic activity for the treatment of cancer, such as a receptor tyrosine kinase inhibitor. Erlotinib is a small molecule epidermal growth factor receptor (EGFR) inhibitor which is conventionally used for treatment of non-small cell lung cancer (NSCLC). Gefitinib is another tyrosine kinase inhibitor which may be formulated in solution and therefore delivered by perispinal administration. This invention includes the use of erlotinib in solution, gefitinib in solution, or an erlotinib or gefitinib derivative or other receptor tyrosine kinase inhibitors, given by perispinal administration for imaging of intracranial malignant tumors, when these imaging agents are radiolabeled or otherwise tagged to facilitate imaging, including for use in patients with lung cancer metastatic to the brain, or metastases to the brain of other malignant tumors which overexpress EGFR, or for treatment of primary brain tumors, including glioblastoma multiforme. Receptor tyrosine kinase is a protein product of the EGFR gene. Inhibition of EGFR-associated tyrosine kinase is a method of treating solid tumors, including NSCLC, and perispinal administration of these agents is a method of the present invention to increase delivery of these agents to intracranial tumors. Erlotinib has a MW of 429. Perispinal administration of the molecules of the present invention leading to delivery of an amount of said molecule to the brain, the eye, or an intracranial tumor effective to facilitate or enable imaging is distinguished from the systemic administration of said molecules.

Multiple Sclerosis. This immune-mediated disease of the brain is conventionally treated by systemic administration of Copaxone® (glatiramer acetate), or interferons, including Avonex®, Rebif®, and Betaseron®. Perispinal administration of radiolabeled versions of these molecules, and radiolabeled versions of other large molecules, including, but not limited to, rituximab, MRA, Intron A®, PEG-Intron®, Infergen®, anti-TNF biologics, anti-IL-1 biologics, monoclonal antibodies directed to myelin-breakdown products and Actimmune® will allow amounts of these large molecules to reach to brain of a human with these disorders sufficient to facilitate or enable functional imaging. In this way disease activity, response to treatment, and disease progression can be established.

Hearing Loss. Hearing loss occurs in humans in many forms. Hearing is essential to the normal conduct of one's daily activities and people with impaired hearing have many difficulties. Hearing loss can date from birth; it can be acquired later in life; or it can be the result of trauma, accident, disease, or a toxic effect of a medication. It can be genetic, either as a solitary disorder or as part of a complex syndrome. Hearing loss is one of the most common chronic neurological impairments, estimated to affect about 4 percent of those under 45 in the United States, and about 29 percent of those 65 years or older.

As defined herein, the neuronal auditory apparatus includes the cochlea, the auditory division of the eighth cranial nerve, and the central auditory pathways. Sensorineural hearing loss is one particular category of hearing loss and is caused by lesions of the cochlea and/or the auditory division of the eighth cranial nerve. Prior to this invention, treatment of this condition was primarily limited to the use of hearing aids.

The pathogenetic mechanism of most forms of hearing loss has yet to be fully defined. The subjects of this patent include central hearing loss due to lesions of the central auditory pathway; sensorineural hearing loss; sudden hearing loss; autoimmune hearing loss; presbycusis; idiopathic hearing loss; and other forms of hearing loss which are not thought to be primarily due to disorders of conduction (such as a ruptured tympanic membrane).

Humans react to sounds that are transduced into neurally conducted impulses through the action of neuroepithelial cells (hair cells) and spiral ganglion cells (neurons) in the inner ear. These impulses are transmitted along the cochlear division of the eighth cranial nerve into the brainstem and the central auditory pathways.

Presbycusis, or age-related hearing loss, is a type of deafness which affects one-third of the population over the age of 75. Presbycusis is known to be associated with neuronal damage, including loss of neuroepithelial (hair) cells and associated neurons (see Schuknecht reference). The exact mechanism of presbycusis is unknown, and has long been thought to be multifactorial. Inflammation is not widely recognized as a significant factor in the pathogenesis of presbycusis. Yet a previous study did suggest that genes encoded by the major histocompatibility complex (MHC) had a role in certain hearing disorders. (Bernstein, Acta Otolaryngol 1996 September; 116(5):666-71). The MHC is known to be central to the immune response and inflammation. Normal hearing is dependant upon proper neuronal function, and may be altered by autoimmune disorders or other types of inflammation. The neuronal auditory apparatus is protected by the blood-brain barrier. Therefore delivery of large molecules to the auditory apparatus by the systemic route is inhibited by the BBB. Delivery of radiolabeled large molecules, in particular anti-TNF biologics, including golimumab and others, or other biologics which reduce inflammation, by perispinal administration, as illustrated herein, is an effective way to image the auditory apparatus when it is inflamed, which occurs in various types of hearing loss, including sensorineural hearing loss and presbycusis.

Neuropsychiatric Disorders. Psychiatric disorders which have a biological basis, such as depression and schizophrenia, can be imaged by the methods of the present invention. In particular, humans with these disorders are amenable to imaging utilizing perispinal administration without direct intrathecal injection of radiolabeled large molecules, including but not limited to anti-TNF molecules, including golimumab and others (as illustrated by the experimental results included herein), MRA (Roche, Chugai), a humanized anti-IL-6 receptor monoclonal antibody; anti-IL-1 molecules; monoclonal antibodies or monoclonal antibody fragments which target serotonin or dopamine receptors, including D1, D2, D3, and D4 receptors, and other dopamine receptor ligands, such as [11C]SCH23390, a commonly used D1 receptor ligand. Imaging is useful for diagnosis, response to treatment, and in drug development.

Disc-related Pain, including low back pain cervical radiculopathy discogenic pain sciatica, and pain associated with degenerative disc disease. The author has considerable experience utilizing perispinal etanercept for the treatment of low back pain, discogenic pain, cervical radiculopathy, sciatica and related disorders which has established the efficacy of this novel method of treatment. Certolizumab pegol or golimumab given to a human or other mammal by perispinal administration is also effective for treating these disorders. Imaging of the spine, spinal cord, nerve roots, dorsal root ganglia, and intervertebral discs with radiolabeled large molecules, including radiolabeled biologic anti-TNF molecules (etanercept, infliximab, certolizumab pegol, golimumab or adalimumab) or radiolabeled anti-IL1 molecules, delivered by perispinal administration, is of diagnostic utility. This method allows the detection of areas of inflammation within the above anatomic structures, and will assist in the identification of the “pain generator” in patients with multiple structural abnormalities at different anatomic levels. This is of significant practical utility, especially in patients in whom clinical examination and routine MRI imaging together cannot definitively identify the source of a patient's chronic back or neck pain and surgical fusion or disc replacement is being contemplated. By identifying the source of the patient's pain the target intervertebral disc or other anatomic structure for surgical intervention can be correctly determined. Alternatively the correct target can be identified by this type of functional molecular imaging for consideration of non-surgical treatment.

### Dosages and Routes of Administration

The therapeutically effective dosage of a large molecule used for perispinal administration will in general be 10% to 100% of the dosage used as a single dose for systemic administration. This dosage used for systemic administration is well known by those skilled in the art as it is specified in the FDA approved literature which accompanies each of these biologics, since each is FDA approved for other clinical uses. For example, if the usual dose when administered systemically is 50 mg, then the dose used for perispinal administration for therapeutic use will usually be between 5 mg and 50 mg. For diagnostic uses of large molecules one has two choices. If one is interested in achieving a therapeutic effect, in addition to facilitating imaging, then the dosing regimen detailed above is utilized. If one is only interested in diagnostic imaging, then microdoses of the radiolabeled large molecule are utilized. A microdose is 1/100 or less of the therapeutic dose, which is designed to be less than 100 micrograms, with doses in the 0.5 to 10 microgram range commonly utilized.

Radiolabeled golimumab may be administered to the perispinal area by interspinous injection at a dose of 2 mg to 10 mg, or microdosed for PET imaging in the range of 0.5 to 100 micrograms.

Radiolabeled certolizumab pegol may be administered to the perispinal area by interspinous injection at a dose of 2 mg to 10 mg, or microdosed for PET imaging in the range of 0.5 to 100 micrograms.

Radiolabeled etanercept may be administered in the perispinal area subcutaneously in the human and the dosage level is in the range of 10 mg to 50 mg per dose. For PET imaging, microdosing of radiolabeled etanercept may be used with dosages in the range of 1 to 100 micrograms.

It will be appreciated by one of skill in the art that appropriate dosages of the compounds, and compositions comprising the compounds, can vary from patient to patient. The determination of the optimal dosage will generally involve the balancing of the level of diagnostic benefit against any risk or deleterious side effects. The selected dosage level will depend on a variety of factors including, but not limited to, the activity of the particular compound, the route of administration, the time of administration, the rate of excretion of the compound, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired imaging effect without causing substantial harmful or deleterious side-effects.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

Definitions provided herein are not intended to be limiting from the meaning commonly understood by one of skill in the art unless indicated otherwise.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

### 6. ADVANTAGES OF THE PRESENT INVENTION

Accordingly, an advantage of the present invention is that it provides for enhanced delivery of a imaging agents to the brain, the retina, the eye, the cranial nerves, the auditory apparatus, the spine, the spinal cord, the spinal nerve roots, the dorsal root ganglia, and/or the prostate utilizing perispinal administration or other forms of local administration to facilitate delivery via the vertebral venous system or its branches for improved methods of diagnosis and/or therapeutic use.

Accordingly, an advantage of the present invention is that it provides improved methods of diagnosis by delivering imaging agents, including large molecule imaging agents, across the blood-brain, blood-eye, and blood-nerve barriers to facilitate, improve, or enable diagnostic imaging.

These methods provide improved methods of diagnosis. In addition, these methods provide advantages for gauging disease progression over time, the stage or extent of disease or disorder, and/or for determining the effectiveness of treatment, particularly for diseases or disorders of the brain, spinal structures, and prostate.

Additional advantages include improved diagnosis of the extent of amyloid deposition in the brain, of brain and spinal tumors, and of areas of inflammation involving the spine and related structures, such as the spinal nerve roots and intervertebral discs.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed is:

1. A method for delivering a radiolabeled molecule to a mammal for diagnosis, comprising administering said radiolabeled molecule parenterally into the perispinal space of said human without direct intrathecal injection.

2. A method for delivering a radiolabeled molecule to a mammal for diagnostic imaging, comprising administering said radiolabeled molecule parenterally into the perispinal space of said human without direct intrathecal injection.

3. A method for delivering radiolabeled trastuzumab to the brain, comprising administering said radiolabeled trastuzumab parenterally into the perispinal space of said human without direct intrathecal injection.

4. A method for delivering a radiolabeled biologic to a human for diagnosis of back pain, comprising administering said radiolabeled biologic parenterally into the perispinal space of said human without direct intrathecal injection.

5. A method for delivering etanercept to a human for diagnostic imaging, comprising the steps of:
a) Radiolabeling etanercept with a PET tracer; and
b) administering said etanercept parenterally into the perispinal space of said human without direct intrathecal injection.

6. The method of claim 2, wherein said mammal is a human.

7. The method of claim 2, wherein said radiolabeled molecule includes etanercept.

8. The method of claim 2, wherein said radiolabeled molecule includes golimumab.

9. The method of claim 2, wherein said radiolabeled molecule includes certolizumab pegol.

10. The method of claim 2, wherein said radiolabeled molecule includes trastuzumab.

11. The method of claim 2, wherein said radiolabeled molecule includes bevacizumab.

12. The method of claim 4, wherein said radiolabeled molecule includes etanercept.

13. The method of claim 4, wherein said radiolabeled molecule includes golimumab.

14. The method of claim 4, wherein said radiolabeled molecule includes certolizumab pegol.

15. The method of claim 5, wherein said diagnostic imaging is for back pain.

16. The method of claim 5, wherein said diagnostic imaging is for pain.

17. The method of claim 5, wherein said diagnostic imaging is for neck pain.

18. The method of claim 5, wherein said diagnostic imaging is for sciatica.

19. The method of claim 5, wherein said diagnostic imaging is for cervical radiculopathy.

20. The method of claim 5, wherein said diagnostic imaging is for discogenic pain.

21. The method of claim 5, wherein said diagnostic imaging is for degenerative disc disease.

22. The method of claim 5, wherein said diagnostic imaging is PET imaging.

23. The method of claim 1, wherein said radiolabeled molecule is delivered to the spine for imaging of the spine for diagnosis of a spinal disorder.

24. The method of claim 1, wherein said radiolabeled molecule is delivered to the brain for imaging of the brain for diagnosis of a brain disorder.

25. The method of claim 1, wherein said radiolabeled molecule is delivered to the cerebrospinal fluid for imaging of the brain for diagnosis of a brain disorder.
